(19) United States
(12) Patent Application Publication
(10) Pub. No.: US 2022/0288001 A1
(43) Pub. Date: Sep. 15, 2022

(54) CREATINE FOR IMMUNOTHERAPY

(71) Applicant: The Regents of the University of California, Oakland, CA (US)

(72) Inventors: Lili Yang, Los Angeles, CA (US); Stefano Di Biase, Los Angeles, CA (US)

(73) Assignee: The Regents of the University of California, Oakland, CA (US)

(21) Appl. No.: 17/763,583

(22) PCT Filed: Sep. 24, 2020
(86) PCT No.: PCT/US2020/052437

§ 371 Date: Mar. 24, 2022

Related U.S. Application Data
(60) Provisional application No. 62/905,661, filed on Sep. 25, 2019.

(51) Int. Cl. A61K 31/198; A61K 31/337; A61K 31/555; C07K 16/28; A61P 35/00

(52) U.S. Cl. CPC A61K 31/198; A61K 31/337; A61K 31/555; C07K 16/2809; C07K 16/2818; A61P 35/00; A61K 2039/505

(57) ABSTRACT

As disclosed herein, we have discovered that creatine is a critical molecule buffering ATP levels in cancer-targeting CD8 T cells through maintaining a readily available high-energy phosphate reservoir. Building upon this discovery, we have designed a number of methods for modulating energy metabolism in a population of tumor-infiltrating CD8 T cells, methods that can be adapted for use in therapeutic regimens for the treatment of cancer. Illustrative embodiments of the invention include methods for enhancing tumor-infiltrating CD8 T cells ability to mount and sustain a response to tumor cells comprising increasing the concentrations of creatine available for tumor-infiltrating CD8 T cells energy metabolism, thereby enhancing the ability of the tumor-infiltrating CD8 T cells to mount and sustain a response to the tumor cells.

### CROSS REFERENCE TO RELATED APPLICATIONS

This application claims the benefit under 35 U.S.C. Section 119(e) of co-pending and commonly-assigned U.S. Provisional Patent Application Ser. No. 62/905,661, filed on Sep. 25, 2019, and entitled “CREATINE FOR IMMUNOTHERAPY” which application is incorporated by reference herein.

### STATEMENT AS TO RIGHTS TO INVENTIONS MADE UNDER FEDERALLY SPONSORED RESEARCH AND DEVELOPMENT

This invention was made with government support under Grant Number CA 196335, awarded by the National Institutes of Health. The Government has certain rights in the invention.

### TECHNICAL FIELD

This disclosure relates to methods and materials useful for modulating CD8 T cell metabolism.

### BACKGROUND OF THE INVENTION

T cells play a central role in mediating and orchestrating immune responses against cancer; therefore, they are attractive therapeutic targets for treating cancer (Baumeister et al., 2016; Couzin-Frankel, 2013; Lim and June, 2017; Page et al., 2014; Ribas, 2015; Rosenberg and Restifo, 2015). The maintenance and activation of T cells are energy-demanding activities, requiring the use of bioenergy in the form of adenosine triphosphate (ATP) (Fox et al., 2005). Distinct metabolic programs are utilized by T cells to generate ATP to support their diverse homeostatic and effector functions (Fox et al., 2005; Kidani and Bensinger, 2017; O'Neill et al., 2016; Zeng and Chi, 2017). In the tumor microenvironment, T cells face the special challenge of competing with fast-growing tumor cells for metabolic fuel like glucose, amino acids, and lipids, which can be limiting (McCarthy et al., 2013). Therefore, an efficient and economical bioenergy metabolism is needed for tumor-infiltrating T cells to mount and sustain effective anticancer responses (Siska and Rathmell, 2015). However, the study of metabolic regulators controlling antitumor T cell immunity has just begun and few methods and materials are available to artisans for controlling antitumor T cell immunity (Chang and Pearce, 2016; Ho and Kaech, 2017; Kishton et al., 2017; Patel and Powell, 2017).

For the reasons noted above, there is a need in the art for methods and materials useful for modulating T cell metabolism, for example, methods and materials that can be used to augment cancer-targeting CD8 T cells in immunotherapeutic techniques.

### SUMMARY OF THE INVENTION

As discussed in detail below, we have discovered that creatine is a critical molecule for buffering ATP levels in cancer-targeting CD8 T cells, one which acts by maintaining a readily available high-energy phosphate reservoir for these cells. We found that tumor-infiltrating immune cells upregulate their expression of the creatine transporter gene (SLC6A8 or Cr7), which encodes a surface transporter protein which controls the uptake of creatine into these cells. In this context we further determined that creatine uptake deficiency severely impairs CD8 T cell responses to tumor challenge in vivo and to antigen stimulation in vitro. We then show that supplementation of creatine in vivo through either direct administration or dietary supplement increases ATP levels in cancer-targeting CD8 T cells and that CD8 T cells antitumor activity is enhanced and cancer cell growth is then concordantly suppressed by these creatine augmented CD8 T cells in multiple mouse tumor models. Notably, the combination of a creatine supplement with chemotherapeutics agents such as those used in checkpoint inhibitor blockade treatment (e.g. a PD-1/PD-L1 blockade), showed superior tumor suppression efficacy, providing strong evidence that creatine supplementation is a valuable component for combination cancer immunotherapies.

Embodiments of the invention disclosed herein harness the discovery that creatine is an important “molecular battery” in CD8 T cells, one that conserves bioenergy to power anti-tumor T cell immunity. The disclosure provided herein therefore illustrates the potential of creatine supplementation as a means to improve T cell-based cancer immunotherapies. The discoveries disclosed herein have been harnessed to design new methods and materials useful to augment cancer-targeting CD8 T cells in immunotherapeutic techniques. For example, embodiments of the invention disclosed herein include immunotherapeutic methods for enhancing the ability of tumor-infiltrating CD8 T cells to mount and sustain a response to tumor cells comprising increasing the concentrations of creatine available for tumor-infiltrating CD8 T cells energy metabolism, thereby enhancing the ability of the tumor-infiltrating CD8 T cells to mount and sustain a response to the tumor cells. Typically, in such methods the tumor-infiltrating CD8 T cells are disposed in an individual diagnosed with cancer and exhibit a selected phenotype such as having upregulated expression of a creatine transporter gene (SLC6A8 or Crt); and/or impeded activation of the TCR proximal signalling molecule Zap70. In certain embodiments of the invention, the individual to whom creatine is administered is undergoing a therapeutic regimen comprising the administration of antitumor agents such as immune checkpoint inhibitors (e.g. immune checkpoint inhibitors selected to affect a PD-1/PD-L1 blockade).

Embodiments of the invention include compositions of matter comprising a creatine, a chemotherapeutic agent, and a pharmaceutically acceptable carrier. Typically, creatine is present in the composition in amounts such that concentrations of creatine available for tumor-infiltrating CD8 T cells are increased by at least 10% or more in an individual administered the composition. In certain embodiments of the invention, the creatine is present in the composition in specific amounts such as at least 100 mg. In some embodiments of the invention, creatine is present in the composition in functional amounts selected so that serum creatine concentrations are increased by at least 25 μM in an individual administered the composition. In certain embodiments of the invention, the chemotherapeutic agent comprises at least one immune checkpoint inhibitor selected to affect a PD-1/PD-L1 blockade. Optionally the chemotherapeutic agent comprises an antibody such as pembrolizumab, nivolumab, atezolizumab, avelumab, bevacizumab, durvalumab and the like. In other embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine or the like.

Another embodiment of the invention is a method of modulating energy metabolism in a population of tumor-infiltrating CD8 T cells comprising increasing amounts of creatine in the environment in which the CD8 T cells are disposed such that increased amounts of creatine are available for tumor-infiltrating CD8 T cell energy metabolism, thereby modulating energy metabolism in the population of tumor-infiltrating CD8 T cells. Typically in these methods, the tumor-infiltrating CD8 T cells exhibit an antigen-experienced phenotype (CD44^{hi}CD62L^{lo}) and are disposed in an individual diagnosed with cancer who is undergoing a therapeutic regimen comprising the administration of chemotherapeutic agents. A related embodiment of the invention is a method of reducing proportions of “exhaustion-prone” phenotype cells (PD-1^{hi}CD62L^{lo}) among a population of tumor-infiltrating CD8 T cells comprising delivering creatine to the tumor-infiltrating CD8 T cells so that the creatine is available for tumor-infiltrating CD8 T cell energy metabolism and the proportion of exhaustion-prone phenotype cells (PD-1^{hi}CD62L^{lo}) among the population of tumor-infiltrating CD8 T cells is thereby reduced.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A-1G: CrT-knockout mice show impeded control of tumor growth. FIG. 1A provides a graph of data showing creatine transporter (CrT or Slc6a8) mRNA expression in spleen (SP) cells and tumor-infiltrating immune cells (TIIs) in a mouse B16-OVA melanoma model (n=3-4) measured by qPCR. Cells were collected on day 14 post-tumor challenge. FIG. 1B provides a diagram showing creatine uptake and creatine-mediated bioenergy buffering in cells with high-energy demand. Cr, creatine; PCr, phospho-creatine; Cm, creatinine; CK, creatine kinase. FIGS. 1C-1G provide data from studies of B16-OVA tumor growth in CrT-WT or CrT-KO littermate mice. FIG. 1C provides a schematic of the study's experimental design. FIG. 1D provides a graph of data showing tumor growth (n=3). (FIG. 1E-FIG. 1G) On day 14, tumors were collected from experimental mice and TIIs were isolated for further analysis. FIG. 1E provides a FACS plots showing the detection of tumor-infiltrating CD4 and CD8 T cells (gated as TCRβ⁺CD4⁺ and TCRβ⁺CD8⁺ cells, respectively). FIG. 1F provides a FACS plot showing PD-1 expression on tumor-infiltrating CD8 T cells. FIG. 1G provides a graph of data showing quantification of F (n=3). Representative of 2 (FIG. 1A) and 3 (FIG. 1C to FIG. 1G) experiments, respectively. Data are presented as the mean±SEM. *P<0.05, **P<0.01, by 1-way ANOVA (A) or by Student's t test (FIG. 1D and FIG. 1G). See also FIG. 8.

FIGS. 2A-2H: Creatine uptake deficiency directly impairs antitumor T cell immunity. B16-OVA tumor growth in BoyJ mice was studied. BoyJ mice received adoptive transfer of OVA-specific OT1 transgenic CD8 T cells that were either wild-type or knockout of CrT gene (denoted as OT1^{CrT-WT} or OT1^{CrT-KO} cells, respectively). FIG. 2A shows the Experimental design. FIG. 2B provides a graph of data showing Tumor growth (n=9). In FIGS. 2C-2H, on day 20, tumors were collected from experimental mice and Tils were isolated for further analysis. FIG. 2C provides FACS plots showing the detection of tumor-infiltrating OT1 T cells (gated as CD45.2⁺CD8⁺ cells). FIG. 2D shows Quantification of C (n=9). FIG. 2E provides FACS plots showing PD-1 expression on tumor-infiltrating OT1 T cells. FIG. 2F shows Quantification of E (n=9). FIG. 2G provides FACS plots showing intracellular IL-2 production of tumor-infiltrating OT1 T cells. Prior to intracellular cytokine staining, TIIs were stimulated with PMA and Ionomycine in the presence of GolgiStop for 4 hours. FIG. 2H shows Quantification of G (n=8). Representative of 2 experiments (FIG. 2A to FIG. 2H). Data are presented as the mean±SEM. ns, not significant, *P<0.05, by Student's t test. See also FIG. 9.

FIGS. 3A-3S: Creatine uptake regulates CD8 T cell response to antigen stimulation. In FIG. 3A-FIG. 3N, CD8 T cells were purified from CrT-WT or CrT-KO mice and stimulated in vitro with plate-bound anti-CD3 (5 μg/ml) (n=3-4). Analysis of CrT mRNA expression is shown in FIG. 3A, CrT protein expression is shown in FIG. 3B, cell proliferation is shown in FIG. 3C, cell survival is shown in FIG. 3D, effector cytokine production is shown in FIG. 3E to 3G and FIG. 3J to FIG. 3L, activation marker expression is shown in FIGS. 3H and 3I, and cytotoxic molecule production is shown in FIGS. 3M and 3N). These were shown, either over a 4 to 5-day time course (3A, 3C, 3D, 3E, and 3J) or 48 hours after anti-CD3 stimulation (FIG. 3F to FIG. 3I, FIG. 3K to FIG. 3N). (FIG. 3O-FIG. 3S) CrT-KO CD8 T cells were stimulated in vitro with anti-CD3 and transduced with a MIG-CrT retrovector (FIG. 3O) (n=3). The analysis of retrovector transduction rate (FIG. 3P), CrT mRNA expression (FIG. 3Q) and IL-2 effector cytokine production (FIG. 3R and FIG. 3S) at 96 hours post-stimulation were shown. Representative of 2 (FIG. 3O to FIG. 3S) and 3 (FIG. 3A to FIG. 3N) experiments, respectively. Data are presented as the mean±SEM. ns, not significant, *P<0.05, **P<0.01, ***P<0.001, ****P<0.0001, by Student's t test. See also FIG. 10.

FIG. 4: Creatine uptake modulates CD8 T cell activation by regulating T cell ATP/energy buffering. FIG. 4A shows a Schematic of creatine-mediated ATP/energy buffering. In FIGS. 4B-4E CrT-WT CD8 T cells were stimulated with anti-CD3 and analyzed for mRNA expression of creatine transporter (CrT; FIG. 4B), Creatine kinase brain form (Ckb; FIG. 4C), and two enzymes controlling the de novo synthesis of creatine, Agat (FIG. 4D) and Gamt (FIG. 4E). N=3-9. A.U., artificial unit relative to Ube2d2. In FIGS. 4F-4G CrT-WT and CrT-KO CD8 T cells were stimulated with anti-CD3 and analyzed for intracellular levels of ATP over time (FIG. 4F), and creatine at 48 hours (FIG. 4G). N=4. In FIGS. 4H-4J, CrT-KO CD8 T cells were stimulated with anti-CD3, with or without ATP supplementation (100 μm) in the culture medium, and analyzed for surface CD25 activation marker expression (FIG. 4H and FIG. 4I) and IFN-γ effector cytokine production (FIG. 4J) at day 3. N=3-6. FIG. 4K shows Western blot analysis of TCR signaling events in CrT-WT and CrT-KO CD8 T cells. CrT-WT and CrT-KO CD8 T cells were stimulated with anti-CD3 for 48 hours, rested at 4° C. for 2 hours, then restimulated with anti-CD3 for 30 minutes followed by western blot analysis. FIG. 4L shows Western blot analysis of TCR signaling events in CrT-KO CD8 T cells with or without ATP supplementation. CrT-KO CD8 T cells were stimulated with anti-CD3 for 48 hours, rested at 4° C. for 2 hours, then restimulated with anti-CD3 for 30 minutes in the presence or absence of ATP supplementation (100 μm) followed by western blot analysis. FIG. 4M shows Western blot analysis of TCR signaling events in CrT-WT and CrT-KO CD8 T cells with or without AICAR treatment. CrT-WT and CrT-KO CD8 T cells were pretreated with AICAR (2 mM) for 30 minutes, then stimulated with anti-CD3 for 20 minutes followed by western blot analysis. AICAR, 5-aminoimidazole-4-carboxamide 1-β-D-ribofuranoside, a stimulator of AMPK; DMSO, dimethylsulfoxide, solvent used to dissolve AICAR. FIG. 4N shows a Schematic model showing creatine uptake regulation of T cell activation signaling events. The demonstrated pathways are highlighted in red and blue. Representative of 2 experiments (4B to 4M). Data are presented as the mean±SEM. *P<0.05, **P<0.01, ***P<0.001, ****P<0.0001, by Student's t test. See also FIG. 11.

FIGS. 5A-5K: Creatine supplementation for cancer immunotherapy. FIGS. 5A-5G show studies of the therapeutic potential of creatine supplementation in a B16-OVA melanoma model. FIG. 5A shows the Experimental design. FIG. 5B shows Creatine levels in serum (n=5). FIG. 5C shows Tumor progression (n=8-10). (D-G) On day 17, tumors and muscles were collected from experimental mice for further analysis. FIG. 5D shows FACS plots showing the phenotype of tumor-infiltrating CD8 T cells. FIG. 5E shows Quantification of FIG. 5D (n=4-6). FIG. 5F shows H&E-stained skeletal muscle sections. Scale bar: 100 μm. FIG. 5G shows Quantification of F (n=3). FIGS. 5H-5I show studies of the requirement of an intact immune system for cancer therapy effects. FIG. 5H shows the Experimental design. FIG. 5I shows Tumor progression (n=5). NSG: NOD/SCID/γc^{-/-} immunodeficient mice. FIGS. 5J and 5K shows studies of the requirement of T cells for creatine cancer therapy effects. I.p. injection of an anti-CD3 depleting antibody (αCD3, clone 17A2) was used for in vivo depletion of T cells. FIG. 5J shows the Experimental design. FIG. 5K shows Tumor progression (n=5-9). Representative of 2 (FIG. 5H to FIG. 5K) and 3 (5A to 5G) experiments, respectively. Data are presented as the mean±SEM. ns, not significant, *P<0.05, **P<0.01, ***P<0.001, ****P<0.0001, by 1-way ANOVA (FIG. 5B, FIG. 5C. FIG. 5E, FIG. 5G, FIG. 5K) or by Student's t test (1). See also FIG. 12.

FIGS. 6A-6E: Creatine supplementation for combination cancer therapy. Studying the therapeutic potential of creatine supplementation in combination with anti-PD-1 (αPD-1) treatment in an MC38 colon cancer model. FIG. 6A provides the Experimental design. FIG. 6B shows Tumor progression at Phase-1 (n=4-5). FIG. 6C shows Tumor progression at Phase-2 (n=3-4). FIG. 6D shows Detection of memory CD8 T cells (gated as CD8′CD44e) in blood of tumor-bearing mice at Phase-2. FIG. 6E shows Quantification of D (n=3-4). Representative of 2 experiments (FIG. 6A to FIG. 6E). Data are presented as the mean±SEM. *P<0.05, **P<0.01. ***P<0.001. ****P<0.0001, by 1-way ANOVA (FIG. 6B) or by Student's t test (FIG. 6E). See also FIG. 12.

FIGS. 7A-7D: The “Hybrid-Engine” model—an updated view of the molecular machinery that powers antitumor T cell immunity. FIG. 7A shows Nutrients that serve as the biofuels, which can be limiting in the tumor microenvironment. FIG. 7B shows The “Hybrid-Engine” model. To analogize the hybrid car, a tumor-targeting CD8 T cell utilizes a “molecular fuel engine”, such as aerobic glycolysis and/or TCA cycle, to convert nutrients/biofuels into bioenergy in the form of ATP, while utilizing creatine as a “molecular battery” to store bioenergy and buffer the intracellular ATP level in order to power T cell antitumor activities. FIG. 7C shows Creatine can be obtained from creatine-rich dietary resources, mainly red meat, poultry, and fish, as well as from dietary supplements. FIG. 7D shows the best cancer therapy benefits would come from clinical intervention by administering creatine to cancer patients following specially designed dosing strategies.

FIGS. 8A-8K: CrT-knockout mice show impeded control of tumor growth, related to main FIG. 1. FIGS. 8A-8H show the Characterization of CrT-Knockout mice. FIG. 8A shows the Breeding strategy for the generation of CrT-KO mice. FIGS. 8B-8H sow the Characterization of CrT-KO mice in comparison with their CrT-WT littermate controls (n=3-4). FIG. 8B shows CrT-KO mice showed reduced body weight. FIG. 8C shows CrT-KO mice contained normal numbers of immune cells proportional to their body weight. FIGS. 8D-8E show that CrT-KO mice showed normal T cell development in thymus. FIG. 8D provides a FACS plot showing the developmental stages of thymocytes defined by CD4/CD8 co-receptor expression. FIG. 8E provides a Quantification of data in 8D. FIGS. 8F-8G show the CrT-KO mice contained normal levels of CD4 and CD8 T cells in the periphery. FIG. 8F provides FACS plots showing the detection of CD4 and CD8 T cells in the periphery. FIG. 8G provides the Quantification of date in 8F. FIG. 8G shows that Peripheral T cells in the CrT-KO mice displayed a normal naïve T cell phenotype (CD25^{lo}CD69^{lo}CD62L^{hi}CD44^{lo}). FACS plots of peripheral blood T cells were shown. 8I-8J show a Study of B16-OVA tumor growth in CrT-WT and CrT-KO littermate mice without creatine supplementation. (I) Experimental design. (J) Tumor growth (n=3). FIG. 8K provides a Study of CrT gene expression in tumor-infiltrating CD8 T cell subsets. B6 mice were inoculated with B16-OVA tumor cells. On day 19, tumor-infiltrating immune cells were isolated and CD8 T cells (pre-gated as CD45.2⁺TCRβ⁺CD8⁺ cells) were sorted into three subsets: PD-1^{lo}, PD-1^{hi}(Tim-3/LAG-3)^{lo}, and PD-1^{hi}(Tim-3/LAG-3)^{hi}. CD8 T cells (gated as CD45.2⁺TCRβ⁺CD8⁺ cells) sorted from the spleen of age-matched, tumor-free B6 mice were included as a control. qPCR analysis of CrT mRNA expression in the indicated CD8 T cells were presented (n=3). Representative of 2 experiments (FIGS. 8A to 8K). Data are presented as the mean±SEM. ns, not significant, *P<0.05, **P<0.01, ***P<0.001, ****P<0.0001, by Student's t test (FIGS. 8B, 8C, 8E, 8G, 8J), or by 1-way ANOVA (FIG. 8K).

FIGS. 9A-9I: Creatine uptake deficiency directly impairs antitumor T cell immunity, related to main FIG. 2. FIGS. 9A-9D shows data Studying B16-OVA tumor growth in BoyJ mice receiving adoptive transfer of bone marrow cells from the CrT-WT or CrT-KO donor mice (denoted as the BMW^{CrT-WT} or BMT^{CrT-KO} mice, respectively). FIG. 9A shows the Experimental design. FIGS. 9A-9C show the Characterization of BMT^{CrT-WT} and BMT^{CrT-KO} mice. CrT deficiency did not impair the capacity of bone marrow cells to reconstitute T cell compartment in BoyJ recipient mice. FIG. 9B shows FACS plots showing the detection of CD4 and CD8 T cells in blood (gated as CD4⁺ and CD8⁺ cells, respectively). FIG. 9C shows Quantification of data from 9C (n=4-6). FIG. 9D shows Tumor growth (n=4-6). FIGS. 9E-9I shows data Studying the anti-tumor capacity of CrT-WT and CrT-KO OT1 transgenic T cells (related to main FIGS. 2A-2H). FIG. 9E shows Breeding strategy for the generation of OT1 transgenic (OT1 Tg) mice deficient in CrT gene (denoted as OT1Tg^{CrT-KO} mice), in contrast to the conventional OT1 Tg mice (denoted as OT/Tg^{CrT-WT} mice). FIG. 9F shows FACS plots showing the isolation of OT1 transgenic T cells (>991% purity, gated as CD4⁺CD8⁺TCR Vα2⁺TCR Vβ5⁺ cells) from OT/Tg^{CrT-WT} mice (denoted as OT1^{CrT-WT} cells) and OT/Tg^{CrT-KO} mice (denoted as OT1^{CrT-KO} cells) using MACS. MACS, magnetic-activated cell sorting. (9G-9I) On day 20 post-tumor inoculation, B16-OVA tumors were collected from experimental mice and TIIs were isolated for further analysis. OT1 transgenic T cells were identified as CD45.2⁺CD8⁺ cells. FIG. 9G provides data Studying the impact of CrT-deficiency on OT1 T cell infiltration into tumor. Representative FACS plots were presented. Compared to OT1^{CrT-WT} cells, OT1^{CrT-KO} cells showed similar levels of tumor infiltration and displayed a similar antigen-experienced phenotype (CD44^{hi}CD62L^{lo}), but exhibited a more exhaustion-prone characteristic, as shown by the higher expression of PD-1. FIGS. 9H-9I provide data Studying the impact of CrT-deficiency on functionality of tumor-infiltrating OT1 T cells. FIG. 9H shows FACS plots showing the measurements of intracellular IFN-γ. Prior to intracellular cytokine staining, TIIs were stimulated with PMA and Ionomycine in the presence of GolgiStop for 4 hours. FIG. 9I shows Quantification of 9H (n=8). Representative of 2 experiments (9A to 9I). Data are presented as the mean±SEM. ns, not significant, *P<0.05, **P<0.01, by Student's t test.

FIGS. 10A-10I: Creatine uptake regulates CD8 T Cell response to antigen stimulation, related to main FIG. 3. FIG. 10A shows Creatine levels in standard T cell culture medium, measured using a Creatine Assay Kit (Abcam). Note FBS was the source of creatine. RPMI, RPMI 1640 medium; FBS, fetal bovine serum. FIGS. 10B-10J provide data from a Study of creatine uptake regulation of antigen-specific CD8 T cell response. OVA-specific 01 transgenic CD8 T cells were isolated from the OT/Tg^{CrT-WT} or OT/Tg^{CrT-KO} mice (denoted as OT1^{CrT-WT} or OT1^{CrT-KO} cells, respectively) and then stimulated in vitro with anti-CD3. FIG. 10B shows a Schematic of the experimental design to isolate OT1^{CrT-WT} and OT1^{CrT-KO} cells for in vitro stimulation. Analysis of cell proliferation is shown in FIG. 10C (n=3), cell viability is shown in FIG. 10D (n=4), effector cytokine production (E and F) (n=6), surface CD25 activation marker expression is shown in FIGS. 10G and 10H (n=4), and cytotoxic molecule production is shown in FIGS. 101 and 10W (n=4) were shown. Data in FIGS. 10E to 10 J were collected at 48 hours post-stimulation. FIGS. 10K-10L provide data from a Study of CrT-KO CD8 T cells transduced with MIG-CrT retrovector (related to main FIGS. 3O-3S). FIG. 10K shows FACS plots showing the intracellular staining of IFN-γ effector cytokine in GFP⁺ CrT-KO CD8 T cells 96 hours after anti-CD3 stimulation and MIG-CrT transduction. FIG. 10L shows Quantification of K (n=3). Representative of 2 experiments (10A to 10L). Data are presented as the mean±SEM. ns, not significant, *P<0.05, **P<0.01, ***P<0.001, ****P<0.0001, by Student's t test.

FIGS. 11A-11D: Creatine uptake regulates CD8 T cell response by regulating T cell ATP/energy buffering, related to main FIG. 4. FIGS. 11A-11B provide data from a Study of CrT-WT CD8 T cell activation with ATP supplementation. CrT-WT CD8 T cells were stimulated with anti-CD3, with or without ATP supplementation (100 μM) in the culture medium, and analyzed for surface CD25 activation marker at 48 hours. FIG. 11A shows FACS plots showing CD25 expression. FIG. 11B shows Quantification of A (n=3). Note ATP supplementation further increased the activation of CrT-WT CD8 T cells. FIGS. 11C and 11D show data from a Study of CrT-WT and CrT-KO CD8 T cell activation with or without AICAR treatment. CrT-WT and CrT-KO CD8 T cells were pretreated with DMSO or AICAR (250 μM) for 30 minutes followed by stimulation with anti-CD3 for 16 hours. AICAR, 5-aminoimidazole-4-carboxamide 1-β-D-ribofuranoside, a stimulator of AMPK; DMSO, dimethylsulfoxide, solvent used to dissolve AICAR FIG. 11C shows CD25 activation marker expression measured using flow cytometry (n=3). FIG. 11D shows IL-2 production measured using ELISA (n=3). FIG. 11E shows a Study of TCR proximal signaling events in CrT-WT and CrT-KO CD8 T cells with or without creatine supplementation. Purified CrT-WT and CrT-KO CD8 T cells were stimulated in vitro with anti-CD3 in the presence or absence of creatine supplementation (0.5 mM) for 48 hours, rested at 4° C. for 2 hours, then restimulated with anti-CD3 in the presence or absence of creatine (0.5 mM) for 10 minutes. Representative western blot images showing the analysis of Zap-70 phosphorylation were presented. Representative of 2 experiments (11A to 11E). Data are presented as the mean±SEM. ns, not significant, *P<0.05, ***P<0.001, ****P<0.0001, by Student's t test (B) or by 1-way ANOVA (11C and 11D).

FIGS. 12A-12B: Creatine supplementation for cancer immunotherapy, related to main FIGS. 5 and 6. FIG. 12A provides data Studying the requirement of T cells for cancer therapy effects (related to main FIGS. 5J and 5K). Anti-CD3 monoclonal antibody (αCD3, clone 17A2) was used for in vivo depletion of T cells. FACS plots were presented showing the depletion of T cells, in particular CD8 T cells (gated as TCRβ⁺CD8⁺), in peripheral blood of experimental mice after receiving i.p. injection of anti-CD3. FIG. 12B provides data Studying creatine transporter (CrT) and creatine kinase brain form (Ckb) mRNA expression in B16-OVA and MC38 tumor cells using qPCR. N=4. A.U., artificial unit relative to Actb. Representative of 2 experiments (12A and 12B). Data are presented as the mean±SEM. ****P<0.0001, by Student's t test.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

T cells demand massive energy to combat cancer; however, the metabolic regulators controlling antitumor T cell immunity have just begun to be unveiled. When studying nutrient usage of tumor-infiltrating immune cells in mice, we detected a sharp increase of the expression of a CrT (Slc6a8) gene, which encodes a surface transporter controlling the uptake of creatine into a cell. Using CrT knockout mice, we showed that creatine uptake deficiency severely impaired antitumor T cell immunity. Supplementing creatine to wildtype mice significantly suppressed tumor growth in multiple mouse tumor models and the combination of creatine supplementation with a PD-1/PD-L1 blockade treatment showed synergistic tumor suppression efficacy. We further demonstrated that creatine acts as a “molecular battery” conserving bioenergy to power T cell activities. Therefore, our results have identified creatine as an important metabolic regulator controlling antitumor T cell immunity, underscoring the potential of creatine supplementation to improve T cell-based cancer immunotherapies.

As discussed in detail below, we have discovered that creatine is a critical molecule buffering ATP levels in cancer-targeting CD8 T cells through maintaining a readily available high-energy phosphate reservoir (Wyss and Kaddurah-Daouk, 2000). We found that tumor-infiltrating immune cells upregulated their expression of the creatine transporter gene (Slc6a8 or CrT), which encodes a surface transporter controlling the uptake of creatine into a cell (Wyss and Kaddurah-Daouk, 2000). Creatine uptake deficiency severely impaired CD8 T cell responses to tumor challenge in vivo and to antigen stimulation in vitro. Importantly, it has been discovered that supplementation of creatine through either direct administration or dietary supplement overcomes impaired CD8 T cell responses associated with low creatine levels, with the result that tumor growth is then suppressed by the creatine augmented CD8 T cells CD8 T cell in multiple mouse tumor models. Notably, the combination of creatine supplementation with a checkpoint inhibitor blockade treatment, such as the PD-1/PD-L1 blockade, showed synergistic tumor suppression effect, providing strong evidence that creatine supplementation is a valuable component for combination cancer immunotherapies. Therefore, our results have identified creatine as an important “molecular battery” that conserves bioenergy to enhance antitumor T cell immunity, underscoring the potential of creatine supplementation to improve T cell-based cancer immunotherapies.

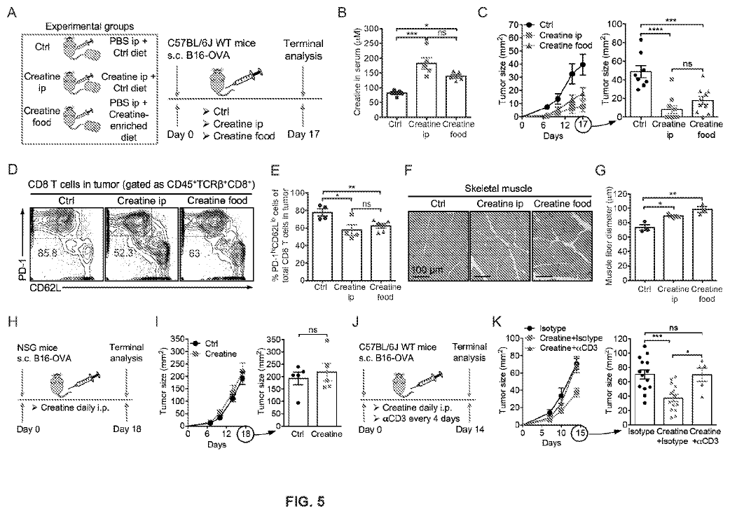

The invention disclosed herein has a number of embodiments. Embodiments of the invention include compositions of matter comprising a creatine, a chemotherapeutic agent, and a pharmaceutically acceptable carrier. In certain embodiments of the invention, the creatine is present in the composition in specific amounts such as at least 100 mg, at least 250 mg, at least 500 mg, at least 1000 mg, at least 5,000 mg, or at least 10,000 mg. However, in view of the fact that different people weigh different amounts and may respond differently to a specific amount of creatine, those of skill in this art understand that a more precise way to describe embodiments of the invention is to include a description of what the composition does (e.g. increase serum creatine concentrations in vivo so that this exogenous creatine can augment CD8 T cell metabolism), rather than by what the composition is (e.g. 100 mg creatine). In this context, artisans understand that creatine is a well-known molecule whose pharmacokinetics etc., are well defined and understood, making the dosing of creatine (e.g. so to increase serum creatine concentrations in vivo by at least a certain amount) routine in this art. See, for example, “Clinical Pharmacology of the Dietary Supplement Creatine Monohydrate” Pharmacological Reviews 2001, 53 (2) 161-176; “Pharmacokinetics of the Dietary Supplement Creatine” 2003, Clinical Pharmacokinetics 42(6):557-74: “Creatine Phosphate: Pharmacological and Clinical Perspectives” Advances in Therapy volume 29, pages 99-123 (2012); Creatine: From Basic Science to Clinical Application (Medical Science Symposia Series) 1st Edition by Rodolfo Paoletti (Editor), A. Poli (Editor), Ann S. Jackson (Editor); as well as U.S. Pat. No. 8,513,306, the contents of each of which are incorporated by reference. Consequently, in certain embodiments of the invention, creatine is present in such compositions in amounts such that concentrations of creatine available for tumor-infiltrating CD8 T cells are increased over existing/endogenous amounts by at least 10%, at least 25%, at least 50% or at least 100% (see, e.g. FIG. 5B) in an individual administered the composition. In some embodiments of the invention, creatine is present in the composition in amounts selected so that serum creatine concentrations are increased by at least 25 μM, at least 50 μM, at least 75 μM or at least 100 μM (see, e.g. FIG. 5B) in an individual administered the composition.

Optionally the chemotherapeutic agent used in the compositions and methods disclosed herein comprises an antibody such as pembrolizumab, nivolumab, atezolizumab, avelumab, bevacizumab, durvalumab and the like. In some embodiments, the chemotherapeutic agent comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine or the like. In certain embodiments of the invention, the chemotherapeutic agent comprises at least one immune checkpoint inhibitor selected to affect a PD-1/PD-L1 blockade.

Embodiments of the invention further include methods of making the compositions of the invention. Such methods include, for example, combining creatine, a chemotherapeutic agent and a pharmaceutically acceptable carrier so that the composition is made. In certain embodiments of the invention, the creatine in the composition is added in specific amounts such as at least 100 mg, at least 250 mg, at least 500 mg, at least 1000 mg, at least 5,000 mg, or at least 10,000 mg. Typically, creatine is added to such compositions in amounts such that concentrations of creatine available for tumor-infiltrating CD8 T cells are increased by at least 10%, at least 25%, at least 50% or at least 100% (see, e.g. FIG. 5B) in an individual administered the composition. In some embodiments of the invention, creatine is added to the composition in amounts selected so that serum creatine concentrations are increased by at least 25 μM, at least 50 μM, at least 75 μM or at least 100 μM (see, e.g. FIG. 5B) in an individual administered the composition.

In certain embodiments of the invention, the chemotherapeutic agent added to the composition comprises at least one immune checkpoint inhibitor selected to affect a PD-1/PD-L1 blockade. Optionally the chemotherapeutic agent added to the composition comprises an antibody such as pembrolizumab, nivolumab, atezolizumab, avelumab, bevacizumab, durvalumab, rituximab and the like. In certain embodiments, the chemotherapeutic agent added to the composition comprises carboplatin, cisplatin, paclitaxel, doxorubicin, docetaxel, cyclophosphamide, etoposide, fluorouracil, gemcitabine, methotrexate, erlotinib, imatinib mesylate, irinotecan, sorafenib, sunitinib, topotecan, vincristine, vinblastine or the like.

The compositions of the invention comprising creatine may be made and then systemically administered in combination with a pharmaceutically acceptable vehicle such as an inert diluent. For oral therapeutic administration, the compounds may be combined with one or more excipients and used in the form of ingestible tablets, buccal tablets, troches, capsules, elixirs, suspensions, syrups, wafers, and the like. For compositions suitable for administration to humans, the term “excipient” is meant to include, but is not limited to, those ingredients described in Remington: The Science and Practice of Pharmacy, Lippincott Williams & Wilkins, 21st ed. (2006) (hereinafter Remington's). Common illustrative excipients include antimicrobial agents and buffering agents.

The compositions of the invention comprising creatine may be administered parenterally, such as intravenously or intraperitoneally by infusion or injection. Solutions of the compositions of the invention comprising creatine can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising compounds which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

Another embodiment of the invention is a method of modulating energy metabolism in a population of tumor-infiltrating CD8 T cells comprising introducing amounts of creatine in the environment in which the CD8 T cells are disposed so that increased amounts of creatine are available for tumor-infiltrating CD8 T cell energy metabolism, thereby modulating energy metabolism in the population of tumor-infiltrating CD8 T cells. Typically in these methods, the tumor-infiltrating CD8 T cells are disposed in an individual diagnosed with a cancer (e.g. a lymphoma or a skin, breast, ovarian, prostate, colorectal or lung cancer) and the individual is undergoing a therapeutic regimen comprising the administration of a chemotherapeutic agent. In certain embodiments of the invention, amounts of creatine are selected to reduce proportions of “exhaustion-prone” phenotype cells (PD-1^{hi}CD62L^{lo}) present in a population of tumor-infiltrating CD8 T cells within the individual. In certain embodiments, amounts of creatine administered to the individual are selected so that concentrations of creatine available for tumor-infiltrating CD8 T cells are increased by at least at least 10%, at least 25%, at least 50% or at least 100%. Optionally, amounts of creatine administered to the individual are selected to be at least at least 100 mg, at least 250 mg, at least 500 mg, at least 1000 mg, at least 5,000 mg, or at least 10,000 mg. In some embodiments of the invention, amounts of creatine administered to the individual are selected so that so that serum creatine concentrations are increased by at least 25 μM, at least 50 μM, at least 75 μM or at least 100 μM (see, e.g. FIG. 5B) in an individual administered the composition.

Embodiments of the invention include methods of treating a cancer in an individual (e.g. a lymphoma or a skin, breast, ovarian, prostate, colorectal or lung cancer), the methods comprising administering the individual creatine in combination with a chemotherapeutic agent. In certain embodiments, the individual is undergoing a therapeutic regimen comprising the administration of at least one immune checkpoint inhibitor selected to affect a PD-1/PD-L1 blockade. In some embodiments of the invention, amounts of creatine administered to the individual are selected to be at least at least 100 mg, at least 250 mg, at least 500 mg, at least 1000 mg, at least 5,000 mg, or at least 10,000 mg. Typically, amounts of creatine administered to the individual are selected so that concentrations of creatine available for tumor-infiltrating CD8 T cells are increased by at least 10%, at least 25%, at least 50% or at least 100% (see, e.g. FIG. 5B) in the individual administered the composition. In some embodiments of the invention, amounts of creatine administered to the individual are selected so that that serum creatine concentrations are increased by at least 25 μM, at least 50 μM, at least 75 μM or at least 100 μM (see, e.g. FIG. 5B) in the individual administered the composition.

Yet another embodiment of the invention is a method of reducing amounts of PD-1^{hi}CD62L^{lo} tumor-infiltrating CD8 T cells among a population of tumor-infiltrating CD8 T cells, the method comprising delivering amounts of creatine to the tumor-infiltrating CD8 T cells so that additional creatine is available for tumor-infiltrating CD8 T cell energy metabolism and amounts of PD-1^{hi}CD62L^{lo} tumor-infiltrating CD8 T cells within the population of tumor-infiltrating CD8 T cells are thereby reduced. Typically in these methods, the PD-1^{hi}CD62L^{lo} tumor-infiltrating CD8 T cells are within an individual diagnosed with cancer. In certain embodiments, the individual is undergoing a therapeutic regimen comprising the administration of at least one immune checkpoint inhibitor selected to affect a PD-1/PD-L1 blockade. In certain embodiments of the invention, amounts of creatine administered to the individual are selected to be at least at least 100 mg, at least 250 mg, at least 500 mg, at least 1000 mg, at least 5,000 mg, or at least 10,000 mg. Typically, amounts of creatine administered to the individual are selected so that concentrations of creatine available for tumor-infiltrating CD8 T cells are increased by at least 10%, at least 25%, at least 50% or at least 100% (see. e.g. FIG. 5B) in the individual administered the composition. In some embodiments of the invention, amounts of creatine administered to the individual are selected so that that serum creatine concentrations are increased by at least 25 μM, at least 50 μM, at least 75 μM or at least 100 μM (see, e.g. FIG. 5B) in the individual administered the composition.

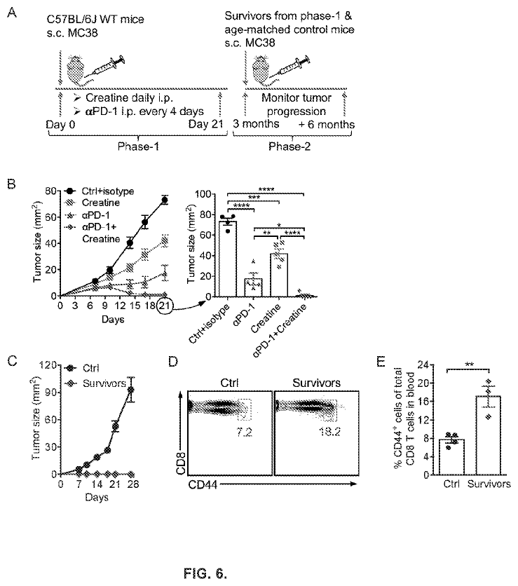

As noted above, embodiments of the invention include the use of immune checkpoint inhibitors. An immune checkpoint inhibitor is a drug—often comprising antibodies—that can facilitate an immune system attack on cancer cells. Such immune checkpoint inhibitors can target, for example, PD-1 (see, e.g. the data presented in FIG. 6) and PD-L1. PD-1 is a checkpoint protein on T cells. PD-1 attaches to PD-L1, a protein on some normal (and cancer) cells. Some cancer cells have large amounts of PD-L1, which helps them evade immune attack. Monoclonal antibodies that target either PD-1 or PD-L1 can block this binding and boost the immune response against cancer cells. Examples of drugs that target PD-1 include: Pembrolizumab (Keytruda) and Nivolumab (Opdivo). These drugs have been shown to be helpful in treating several types of cancer, including melanoma of the skin, non-small cell lung cancer, kidney cancer, bladder cancer, head and neck cancers, and Hodgkin lymphoma. They are also being studied for use against many other types of cancer. Examples of drugs that target PD-L1 include: Atezolizumab (Tecentriq), Avelumab (Bavencio) and Durvalumab (Imfinzi). These drugs have also been shown to be helpful in treating different types of cancer, including bladder cancer, non-small cell lung cancer, and Merkel cell skin cancer (Merkel cell carcinoma). They are also being studied for use against other types of cancer. Other immune checkpoint inhibitors can target other molecules such as cytotoxic T-lymphocyte-associated antigen 4 (CTLA-4), (e.g. ipilimumab (Yervoy®)).

Many of the aspects of the techniques and procedures described or referenced herein are well understood and commonly employed by those skilled in the art. The following provides a description of number of aspects and embodiments of the invention.

### Creatine Transporter (Cr1) Gene is Upregulated in Tumor-Infiltrating Immune Cells

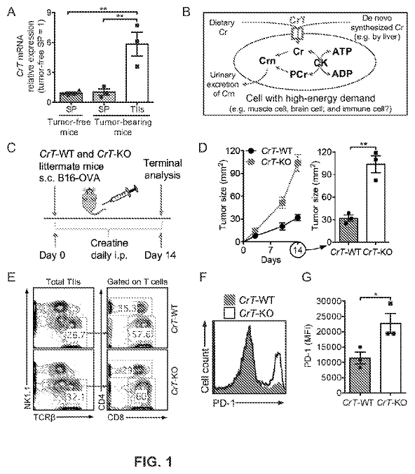

To identify metabolic regulators controlling tumor-fighting immune cells, we grew solid B16-OVA melanoma tumors in C57BL/6J mice, isolated tumor-infiltrating immune cells (TIIs), and then studied their gene expression profile relevant to nutrient usage using quantitative RT-PCR (qPCR). Immune cells isolated from the spleen of tumor-bearing or tumor-free mice were included as controls. Interestingly, in addition to the change of genes involved in the classical glucose/lipid/amino acid metabolic pathways (Fox et al., 2005), we also detected a sharp increase of the expression of a CrT (Slc6a8) gene in TIIs (FIG. 1 A). CrT is an X-linked gene encoding a surface transporter (creatine transporter, CrT) that controls the uptake of creatine into a cell in an Na⁺/K⁺-dependent manner, where creatine is used to store high-energy phosphates and to buffer intracellular ATP levels through a CK/PCr/Cr (creatine kinase/phospho-creatine/creatine) system (FIG. 1 B) (Wyss and Kaddurah-Daouk, 2000). Creatine is a nitrogenous organic acid that naturally occurs in vertebrates. It is mainly produced in the liver and kidneys, but predominantly stored in skeletal muscle (Wyss and Kaddurah-Daouk, 2000). For humans, diet is also a major source of creatine (Wyss and Kaddurah-Daouk, 2000). Expression of CrT is important for cells demanding high-energy like muscle cells and brain cells, in humans, CrT deficiency has been associated with muscle diseases and neurological disorders (Wyss and Kaddurah-Daouk, 2000). On the other hand, oral creatine supplements have been broadly used by bodybuilders and athletes to gain muscle mass and to improve performance (Kreider et al., 2017). However, the function of CrT/creatine outside of the muscle and brain tissues is largely unknown. Since we found upregulated CrT gene expression in TIIs, we asked if the CrT/creatine system might also regulate the energy metabolism of tumor-fighting immune cells, in particular CD8 cytotoxic T cells, which have a massive demand for energy and can benefit from an energy storage/ATP buffering system (FIG. 1 B).

### CrT-Knockout Mice Show Impeded Control of Tumor Growth

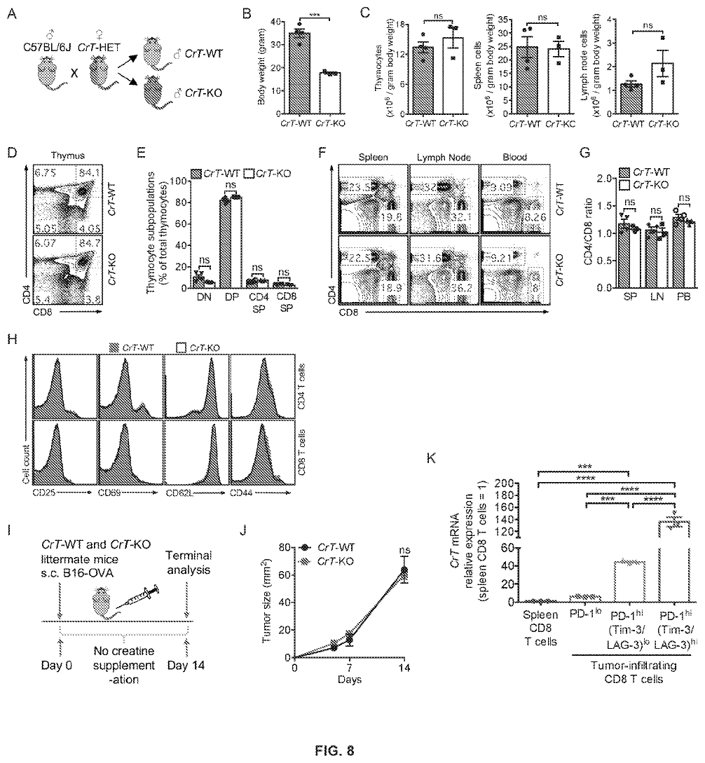

To address this question, we began by studying CrT-Knockout (CrT-KO) mice (FIG. 8 A) (Skelton et al., 2011). Despite their smaller body size. CrT-KO mice contained normal numbers of immune cells, including T cells, proportional to their body weight (FIG. 8, B-G). Prior to tumor challenge, these T cells displayed a typical naïve phenotype (CD25^{lo}CD69^{lo}CD62L^{hi}CD44^{lo}) (FIG. 8 H). In a B16-OVA melanoma model, tumor growth was accelerated in CrT-KO mice compared to that in their CrT-wild-type (CrT-WT) littermates (FIGS. 1, C and D). In CrT-KO mice, tumor-infiltrating CD8 T cells expressed higher levels of PD-1 that has been associated with bioenergy insufficiency and T cell exhaustion, indicating that CrT deficiency may impact antitumor T cell activities (FIG. 1, E-G) (Bengsch et al., 2016; Chang et al., 2015; Wherry and Kurachi, 2015). Of note, the regular mouse diet (PicoLab Rodent Diet 20) does not contain creatine; therefore, in order to mimic the supply of creatine from dietary resources in humans, we supplied creatine to experimental mice via i.p. injection (FIG. 1 C). Without i.p. injection of creatine, no B16-OVA tumor growth difference was observed between CrT-WT and CrT-KO mice, likely due to the lack of sufficient creatine supply in these experimental mice to read out the creatine uptake difference between CrT-WT and CrT-KO mice (FIGS. 8, I and J). Interestingly, study of CrT gene expression in tumor-infiltrating wild-type CD8 T cell subsets showed an upregulation of CrT gene expression that was more significant in the PD-1^{hi} subset than that in the PD-1^{lo} subset, providing evidence of a possible feedback loop in PD-1^{hi} CD8 T cells that compensates for bioenergy-insufficiency by increasing creatine uptake (FIG. 8 K). In particular, the PD-1^{lo}Tim-3^{hi}LAG-3^{hi} tumor-infiltrating CD8 T cells, that are considered to be the most “exhausted”, expressed the highest levels of CrT, providing evidence that these cells may also benefit the most from creatine supplementation treatment (FIG. 8 K) (Nguyen and Ohashi, 2015; Wherry and Kurachi, 2015).

### Creatine Uptake Deficiency Directly Impairs Antitumor T Cell Immunity

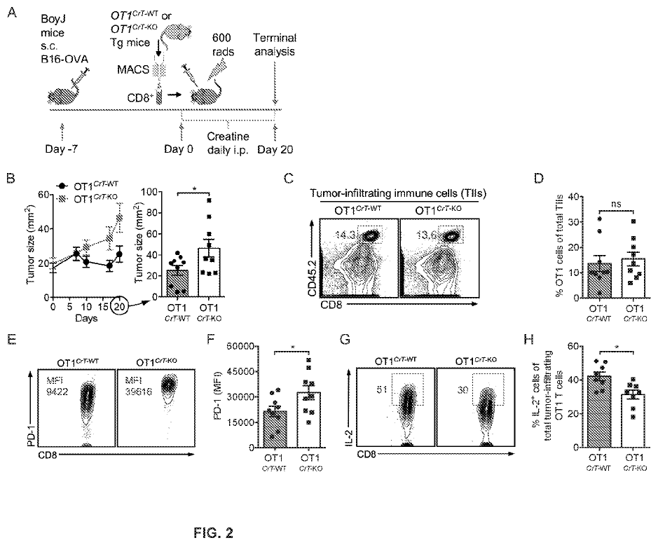

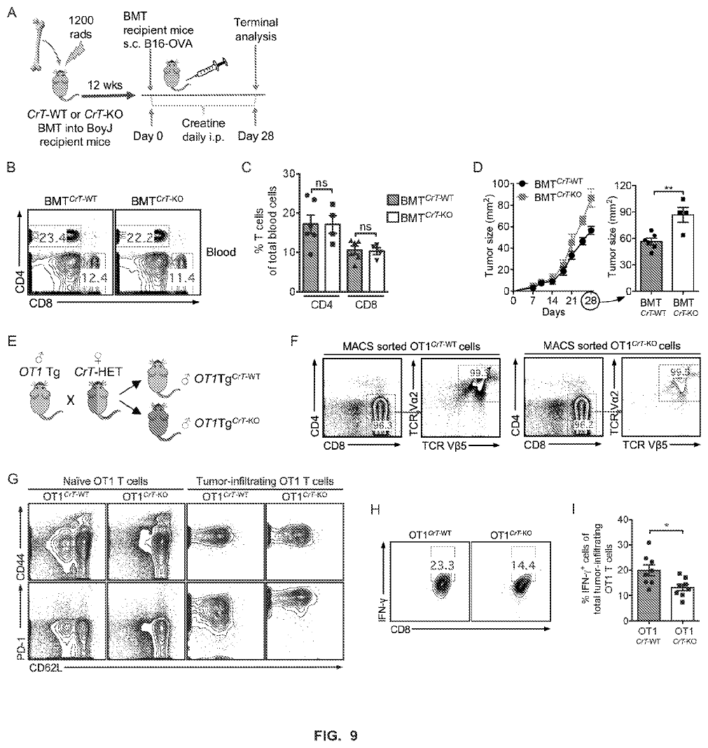

To study the direct regulation of immune cells by CrT, we reconstituted WT BoyJ mice with bone marrow cells from either CrT-WT or CrT-KO donor mice and then challenged recipient mice with B16-OVA tumor cells (FIG. 9 A). CrT-deficiency did not impair the reconstitution of an immune system in the recipient mice (FIGS. 9, B and C), but it did impede the capacity of the reconstituted immune system to control tumor growth (FIG. 9 D). To further study the direct regulation of tumor-specific CD8 T cells by CrT, we bred CrT-KO mice with 071 transgenic (Tg) mice and generated OT/Tg^{CrT-KO} mice producing OVA-specific CD8 T cells deficient in CrT (FIG. 9 E). We isolated OT1^{CrT-WT} and OT1^{CrT-KO} CD8 T cells (FIG. 9 F) and separately transferred these T cells into BoyJ WT mice bearing pre-established B16-OVA tumors (FIG. 2 A). Compared to OT1^{CrT-WT} cells, OT1^{CrT-KO} cells were less effective in controlling tumor growth (FIG. 2 B). Although OT1^{CrT-KO} cells infiltrated tumors and showed an antigen-experienced phenotype (CD62L^{lo}CD44^{hi}) (FIGS. 2, C and D and FIG. 9 G), these T cells expressed higher levels of PD-1 (FIGS. 2, E and F, and FIG. 9 G) and produced less amount of effector cytokines including IL-2 (FIGS. 2, G and H) and IFN-γ (FIG. 9, H and 1) compared to OT1^{CrT-WT} cells. Similarly, mice in these tumor experiments received i.p. injection of creatine to compensate for the lack of creatine supply from mouse diet (FIG. 1 C, FIG. 9 A, and FIG. 2 A). Collectively, these in vivo data demonstrate that creatine uptake deficiency directly impairs antitumor immunity, especially the antitumor efficacy of tumor antigen-specific CD8 cytotoxic T cells.

### Creatine Uptake Regulates CD8 T Cell Response to Antigen Stimulation

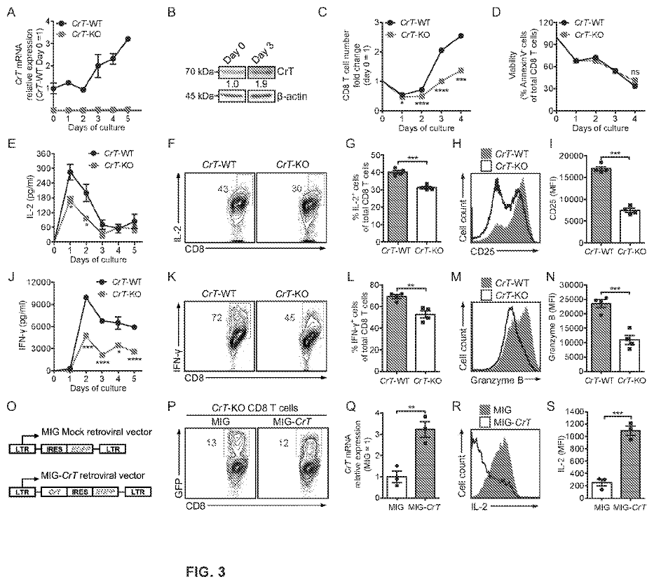

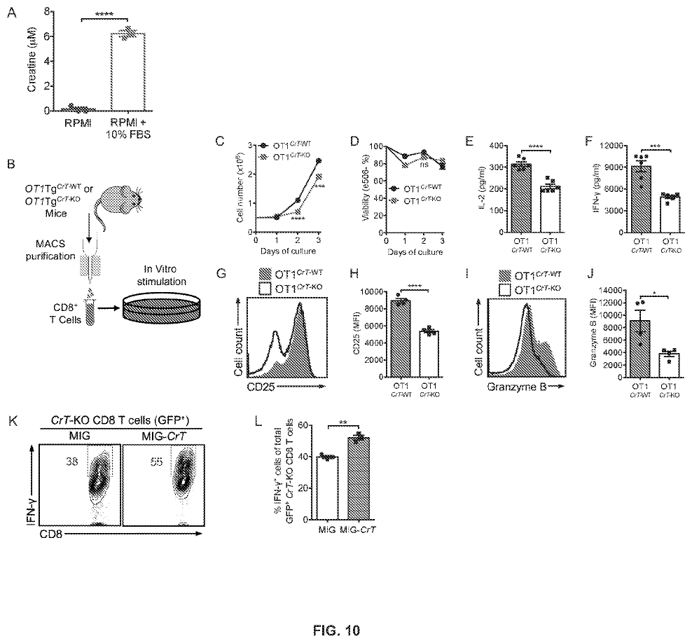

Next, to study how creatine uptake regulates CD8 T cell response to antigen stimulation, we isolated CD8 T cells from CrT-WT or CrT-KO littermate mice, followed by stimulating these cells in vitro with anti-CD3. A standard T cell culture medium was utilized, which comprised 10% FBS as the source of creatine (FIG. 10 A). Post-stimulation. WT CD8 T cells showed upregulated expression of CrT mRNA (FIG. 3 A) and CrT protein (FIG. 3 B), indicating the induction of CrT expression by T cell receptor (TCR) signaling and providing evidence, in turn, for the need for activated CD8 T cells to uptake more creatine. Compared to their CrT-WT counterparts, CrT-KO CD8 T cells showed a reduction in almost all aspects of T cell activation, including cell proliferation (FIG. 3 C), effector cytokine production (e.g., IL-2 and IFN-γ; FIGS. 3, E-G and J-L), surface activation marker expression (e.g., CD25; FIGS. 3. H and I), and cytotoxic molecule production (e.g., Granzyme B; FIGS. 3, M and N). Cell survival, studied via Annexin V and 7-AAD staining, was not affected over a 4-day cell culture period (FIG. 3 D). Study of OVA-specific OT1^{CrT-KO} CD8 T cells gave similar results (FIG. 10, B-J), providing evidence for a general role of CrT in regulating CD8 T cells of diverse antigen specificities. To verify whether creatine uptake deficiency directly contributed to the hyporesponsiveness of the CrT-KO CD8 T cells, we conducted a rescue experiment. We constructed a MIG-CrT retroviral vector (FIG. 3 O), utilized this vector to transduce CrT-KO CD8 T cells, and finally achieved overexpression of CrT in these cells (FIGS. 3, P and Q). CrT overexpression rescued the activation of CrT-KO CD8 T cells and improved their production of multiple effector cytokines (FIGS. 3, R and S; and FIGS. 10, K and L). Taken together, these data indicate that CD8 T cells, post-antigen stimulation, increase their capacity to uptake creatine that is critical for them to manifest a productive effector T cell response.

### Creatine Uptake Modulates CD8 T Cell Activation by Regulating T Cell ATP/Energy Buffering

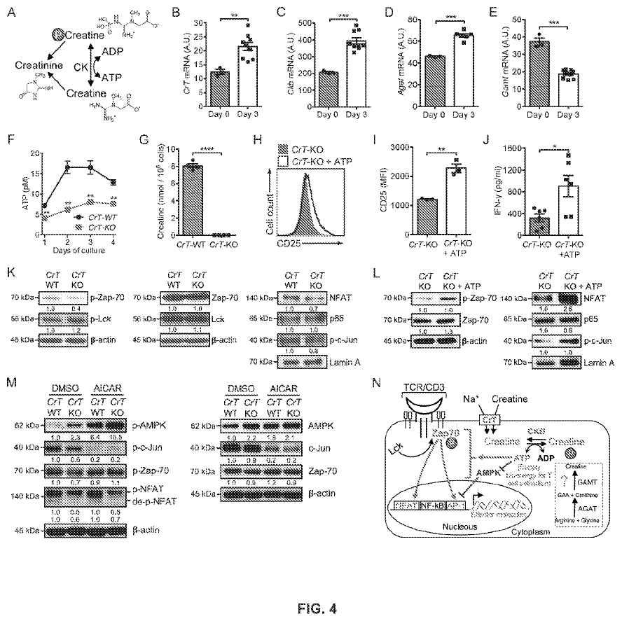

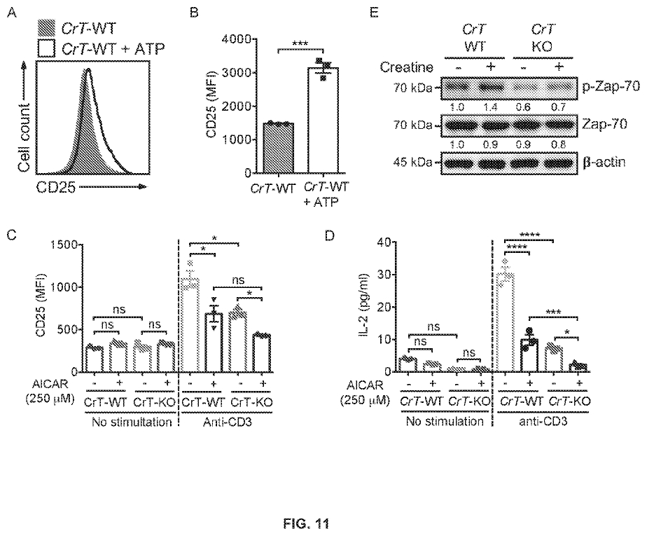

It has been well-characterized that muscle cells and brain cells uptake creatine through CrT and then utilize creatine to buffer intracellular ATP levels and power cellular activities via a CK/PCr/Cr system (Wyss and Kaddurah-Daouk, 2000). Therefore, we investigated whether CD8 T cells might use a similar molecular mechanism (FIG. 4 A). Post-TCR stimulation, WT CD8 T cells upregulated CrT gene expression, enabling the activated T cells to more effectively uptake creatine (FIG. 4 B). CD8 T cells expressed high basal levels of Ckb (creatine kinase brain form) gene, the expression of which was further upregulated post-TCR stimulation, maximizing the capacity of activated CD8 T cells to utilize the CK/pCr/Cr ATP buffering system (FIG. 4 C). De novo synthesized creatine might be another source to feed the CK/pCr/Cr system. Consequently, we examined the expression of genes encoding the two enzymes controlling creatine biosynthesis, Agat (L-arginine:glycine amidinotransferase) and Gamt (guanidinoacetate N-methyltransferase). We found that CD8 T cells expressed low levels of both genes and further downregulated the expression of Gamt gene post-TCR stimulation (FIGS. 4, D and E). Therefore, activated CD8 T cells may have limited capacity to synthesize creatine de novo and may, therefore, heavily rely on importing creatine via CrT from extracellular sources to feed the CK/PCr/Cr ATP-buffering system. In agreement with this notion, compared to CrT-WT CD8 T cells, activated CrT-KO CD8 T cells contained undetectable levels of intracellular creatine (FIG. 4 G) and significantly reduced ATP (FIG. 4 F) (Wyss and Kaddurah-Daouk, 2000). The hypoactivation of CrT-KO CD8 T cells was rescued by supplementing ATP in T cell culture, evidenced by increased expression of T cell surface activation marker CD25 and enhanced production of effector cytokine IFN-γ (FIG. 4, H-J). Supplementing ATP further enhanced the activation of CrT-WT CD8 T cells (FIGS. 11, A and B). ATP supplies bioenergy and phosphate group for TCR signaling events (Patel and Powell, 2017). By comparing the major TCR signaling pathways in CrT-WT and CrT-KO CD8 T cells, we found that creatine uptake deficiency impeded activation of the TCR proximal signaling molecule Zap-70 (zeta chain of T cell receptor associated protein kinase 70) and the downstream transcription factors NFAT (nuclear factor of activated T cells) and c-Jun (Jun proto-oncogene, AP-1 transcription factor subunit), which, at least partially, accounted for the hypoactivation of CrT-KO CD8 T cells (FIG. 4 K). Creatine supplementation significantly increased Zap-70 phosphorylation in CrT-WT CD8 T cells but not in CrT-KO CD8 T cells (FIG. 11 E). The TCR signaling deficiencies in CrT-KO CD8 T cells were effectively rescued by supplementing ATP to the T cell culture (FIG. 4 L). Interestingly, compared to the activation of NFAT and AP-1, the activation of NF-κB (nuclear factor kappa-light-chain-enhancer of activated B cells) in particular its p65 subunit was less sensitive to CrT deficiency-induced ATP shortage, providing evidence that NF-κB signaling pathway may resist better to ATP fluctuation during T cell response (FIGS. 4, K and L). AMPK (5′ adenosine monophosphate-activated protein kinase) is an enzyme that detects shifts in the AMP:ATP ratio within a cell. It serves as a nutrient and energy sensor to maintain cell energy homeostasis, and has been indicated to regulate T cell metabolism and function (Hardie et al., 2012; Ma et al., 2017; Rao et al., 2016; Tamas et al., 2006). We therefore examined the possible role of AMPK in mediating the CrT-KO CD8 T cell hypoactivation phenotype. In correspondence with the decreased ATP levels in CrT-KO CD8 T cells, we detected increased activation of AMPK in these cells compared to that in CrT-WT CD8 T cells (FIGS. 4, F and M). Treating CrT-WT and CrT-KO CD8 T cells with AICAR (5-aminoimidazole-4-carboxamide 1-β-D-ribofuranoside; an AMPK activator) markedly activated AMPK in both T cells (FIG. 4 M), that was associated with a significant reduction of AP-1 transcription factor activation (c-Jun subunit; FIG. 4 M), cell surface activation marker expression (CD25; FIG. 11 C), and effector cytokine production (IL-2; FIG. 11 D) in both T cells. Meanwhile, the activation of Zap-70 and NFAT were not affected by AICAR treatment (FIG. 4 M). Hence, creatine uptake modulation of bioenergy homeostasis in CD8 T cells may be monitored and regulated by AMPK, at least partly through AMPK regulation of the AP-1 pathway. Collectively, these results support an intriguing working model in which activated CD8 T cells 1) employ a potent creatine-mediated ATP/energy buffering system to sustain TCR signaling and power T cell effector functions, at least partly through ATP/AMPK regulation of TCR signaling pathways, and 2) rely on importing creatine via CrT from extracellular sources (FIG. 4 N).

### Creatine Supplementation for Cancer Immunotherapy

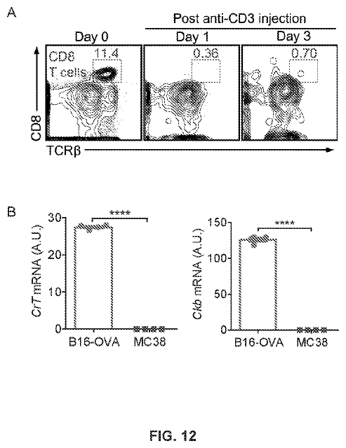

The “creatine-uptake/energy-buffering” working model (FIG. 4 N) opens up the possibility of reinvigorating disease-responding CD8 T cells, in particular tumor-fighting CD8 T cells, through creatine supplementation. To test this new concept of metabolic reprogramming and cancer immunotherapy, we supplemented creatine to experimental C57BL/6J WT mice in the B16-OVA melanoma model, either through i.p. injection or through dietary supplementation (FIG. 5 A). Notably, the dietary supplemental dose we used (0.4 g/kg body weight) is comparable to the safe loading dose recommended for athletes (Kreider et al., 2017). Both administration routes elevated creatine concentrations in blood to a similar level (FIG. 5 B) and effectively suppressed tumor growth to a similar extent (FIG. 5 C). The tumor suppression effect was associated with a significant reduction of the “exhaustion-prone” phenotype cells (gated as PD-1^{hi}CD62L^{lo}) among the tumor-infiltrating CD8 T cells (FIGS. 5, D and E). In agreement with the muscle enhancement effect of creatine, we observed an enlargement of skeletal muscle fibers in mice receiving creatine supplements (FIGS. 5, F and G) (Kreider et al., 2017; Wyss and Kaddurah-Daouk, 2000). On the other hand, B16-OVA tumors grown in immunodeficient NSG mice (FIG. 5, H and 1) or in C57BL/6J WT mice depleted of T cells via i.p. injection of an anti-CD3 depletion antibody (FIGS. 5, J and K; and FIG. 12 A) could not be suppressed by creatine supplementation, confirming that the therapeutic effect of creatine supplementation is mediated by immune cells, in particular T cells. Taken together, these results demonstrate the capacity of creatine supplementation to boost antitumor T cell immunity, thus providing evidence for its potential as a new means of cancer immunotherapy.

### Creatine Supplementation for Combination Cancer Therapy

Many successful and in-development cancer immunotherapies target metabolic reprogramming of immune response in the tumor microenvironment (Ho and Kaech, 2017; Kishton et al., 2017; McCarthy et al., 2013; Patel and Powell, 2017). In particular, checkpoint blockade therapies, such as PD-1/PD-L1 blockade therapies, have been indicated to correct the glucose usage imbalance between tumor cells and T cells by altering glycolysis and directing the energy metabolism to favor T cells (Baumeister et al., 2016; Bengsch et al., 2016; Chang et al., 2015; Gubin et al., 2014; Scharping et al., 2016). By providing a potent and non-redundant energy buffering benefit for tumor-fighting T cells, we postulate that creatine supplementation may synergize with a PD-1/PD-L1 blockade therapy to further improve cancer treatment efficacy. Indeed, in a mouse MC38 colon cancer model sensitive to PD-1/PD-L1 blockade therapy (Homet Moreno et al., 2016), the combination of creatine supplementation and anti-PD-1 treatment generated a significant tumor suppression effect superior to that of each treatment alone (FIGS. 6, A and B). In fact, most experimental mice receiving the combination therapy (4 out of 5) completely eradicated their tumor burden and remained tumor-free for over three months (FIG. 6 C). When receiving a second challenge of MC38 tumor cells, all these “cancer survivors” were protected from tumor recurrence and stayed tumor-free for another 6 months for the duration of the experiment (FIG. 6 C). This appealing tumor protection effect was associated with a significant increase of memory-phenotype CD8 T cells in the surviving mice, most likely generated from the successful antitumor T cell response in the initial tumor challenge and later on utilized by the surviving mice to fight off a second tumor challenge (FIGS. 6, D and E). Collectively, these encouraging results suggest a promising potential of creatine supplementation for combination cancer immunotherapy.

### Discussion

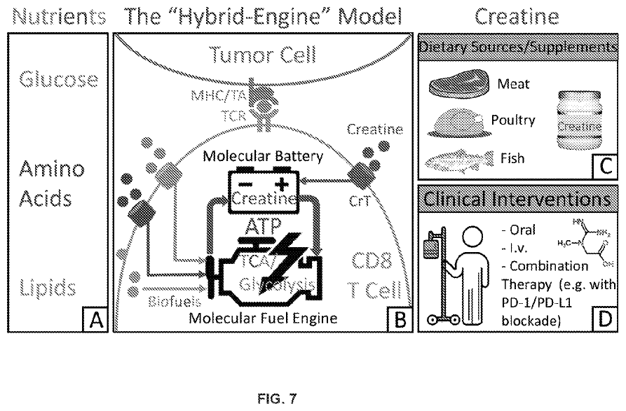

Based on our findings, we propose a “hybrid engine model” to update the molecular machinery that powers antitumor T cell immunity by incorporating creatine into the picture (FIG. 7). Analogous to the popular hybrid car, which uses two distinct sources of power, a tumor-targeting CD8 T cell utilizes a “molecular fuel engine” like glycolysis and/or tricarboxylic acid (TCA) cycle to convert nutrients/biofuels (e.g. glucose, amino acids, and lipids) into bioenergy in the form of ATP, while utilizing creatine as a “molecular battery” to store bioenergy and buffer the intracellular ATP level, in order to support T cell antitumor activities (FIG. 7 B). This “hybrid engine” system is energy-efficient, enabling a tumor-targeting CD8 T cell to make maximal use of its available bioenergy supply and perform in a metabolically stressful microenvironment where it has to compete with fast-growing tumor cells for a limited supply of nutrients (FIG. 7 A) (Fox et al., 2005; Siska and Rathmell, 2015; Wherry and Kurachi, 2015). CD8 T cells have limited capacity to de novo synthesize creatine; therefore, they heavily rely on uptake of creatine from extracellular resources via creatine transporter, CrT (FIG. 7 B), all of which opens up the possibility of reinvigorating tumor-fighting CD8 T cells through creatine supplementation. Creatine can be obtained from creatine-rich dietary resources, mainly red meat, poultry, and fish, as well as from dietary supplements (Kreider et al., 2017; Wyss and Kaddurah-Daouk, 2000) (FIG. 7 C). However, the best cancer therapy benefits would come from clinical intervention by administering creatine to cancer patients following specially designed dosing strategies (FIG. 7 D). Both oral and direct administration (e.g., i.v.) routes can be effective (FIG. 7 D).

Our study showed that creatine supplementation suppressed tumor growth in multiple mouse tumor models, including the B16 melanoma model (FIG. 5) and the MC38 colon cancer model (FIG. 6), providing evidence that this treatment may provide a general therapeutic benefit to many different types of cancer. Moreover, because creatine works through a novel “energy-buffering” mechanism that is non-redundant to the mechanisms used by many successful and in-development immunotherapies, creatine supplementation can potentially become an effective and economical common component for combination cancer immunotherapies. In our study, we showed that creatine supplementation synergized with checkpoint blockade therapies like the PD-1/PD-L1 blockade therapy to yield superior therapeutic efficacy (FIG. 6). Many other cancer therapeutic modalities, including the booming new immunotherapies as well as traditional chemo and radiation therapies, may also benefit from combining with creatine supplementation treatment (FIG. 7 D) (Baumeister et al., 2016; Couzin-Frankel, 2013; Lim and June, 2017; Page et al., 2014; Pardoll, 2012; Ribas, 2015; Rosenberg and Restifo, 2015).

In the past three decades, oral creatine supplements have been broadly utilized by bodybuilders and athletes to gain muscle mass and to improve performance (Kreider et al., 2017; Wyss and Kaddurah-Daouk, 2000). The new discovery that creatine supplementation may help build a stronger immune system in addition to building a stronger body is exciting. For the active users of creatine supplements, this discovery means possible additional health benefits; for disease patients, it means new immunotherapeutic opportunities. The well-documented safety of long-term creatine supplementation in humans affords a “green light” for utilizing creatine supplementation to treat chronic diseases like cancer (Kreider et al., 2017). Meanwhile, the muscle enhancement effect of creatine supplementation, as demonstrated from human experience and shown in our animal studies (FIGS. 5, F and G), may also benefit cancer patients who at their late stages oftentimes suffer from cachexia, or wasting syndrome (de Campos-Ferraz et al., 2014). Interestingly, some early studies showed that creatine and creatine analogues could directly inhibit cancer growth, presumably through disrupting cancer cell metabolism, providing evidence for an additional mechanism that creatine may employ to mediate its antitumor effects (Kristensen et al., 1999; Miller et al., 1993). Conversely. CrT has been suggested as a possible biomarker for circulating tumor cells within the blood, posing the concern that creatine supplement may have potential negative effects on CrT-positive tumors (Riesberg et al., 2016). Interesting, for the two mouse tumor models used in our study, B16 melanoma cells express CrT (as well as CKB) while MC38 colon cancer cells do not (FIG. 12 B). Creatine supplementation exhibited tumor suppression benefits in both tumor models (FIG. 5 C and FIG. 6 B), providing evidence that this therapy has the potential to treat both CrT-positive and CrT-negative tumors.

The “energy-buffering” function of creatine certainly goes beyond regulating CD8 T cells. In CrT-KO mice, we have observed the hyporesponsiveness of multiple immune cells in various mouse tumor models. It is also likely that creatine regulates immune reactions to multiple diseases beyond cancer, such as infections and autoimmune diseases (Riesberg et al., 2016). Studying the roles of creatine in modulating various immune cells under different health and disease conditions will be interesting topics for future research.

### Materials and Methods

### Mice

C57BL/6J (B6) and B6.SJL-Ptprc^{a}Pepc^{b}/BoyJ (CD45.1, BoyJ) mice were purchased from the Jackson Laboratory and six- to ten-week-old mice were used for all the experiments, unless otherwise indicated.

Creatine transporter knockout mice B6(Cg)-Slc6a8^{tm1.2Clar}/J, referred to as the CrT-KO mice, were purchased from the Jackson Laboratory (Skelton et al., 2011). The experimental colony was produced by breeding female hemizygous with male wildtype littermates. Six- to ten-week-old mice were used for all experiments, unless otherwise indicated.

C57BL/6-Tg(TcraTcrb)1100Mjb/J (OT1 Tg) mice were purchased from the Jackson Laboratory and bred with the CrT-KO mice to generate OT/Tg^{CrT-WT} and OT/Tg^{CrT-KO} mice. Six- to ten-week-old mice were used for all the experiments, unless otherwise indicated.

NOD.Cg-Prkdc^{SCID}Il2rg^{tmlWjl}/SzJ (NOD/SCID/IL-2Rγ^{-/-}, NSG) mice were purchased from the Jackson Laboratory. Six- to ten-week old females were used for all experiments, unless otherwise indicated.

The animals were housed under specific pathogen-free conditions with 12-hour day/light cycles. All animal experiments were approved by the Institutional Animal Care and Use Committee (IACUC) of the University of California, Los Angeles (UCLA).

### Antibodies and Flow Cytometry

Fluorochrome-conjugated monoclonal antibodies specific for mouse CD45.2 (cat #109820; clone 104), TCRβ (cat #109220; clone H57-597), CD4 (cat #100531; clone RM4-5), IFN-γ (cat #505806; clone XMG1.2), Granzyme B (cat #372208; clone QA16A02). TCR Vα2 (cat #127809; clone B20.1), CD69 (cat #104508; clone H1.2F3), CD25 (cat #102006; clone PC61), CD8 (cat #100732: clone 53-6.7), CD44 (cat #103030; clone IM7), LAG-3 (CD223) (cat #125207; clone C9B7W), and Tim-3 (CD366) (cat #119705; clone RMT3-23) were purchased from BioLegend. Monoclonal antibodies specific for mouse IL-2 (cat #554428; clone JES6-5H4); TCR Vβ5 (cat #1553190; clone MR9-4); and Fc block (anti-mouse CD16/32) (cat #553142; clone 2.4G2) were purchased from BD Biosciences. Monoclonal antibody specific for mouse PD-1 (cat #12-9981-83; clone RMPI-30) was purchased from the eBioscience. Fixable Viability Dye eFluor 506 (cat #65-0866) was purchased from Thermo Fisher Scientific. Cells were stained with Fixable Viability and Fc blocking dye first, followed by surface marker staining. To detect intracellular molecules (Granzyme B and cytokines), cells were subjected to intracellular staining using a Cell Fixation/Permeabilization Kit (Cat #554714, BD Biosciences), following the manufacturer's instructions. To analyze cell viability, cells were stained with Annexin V and 7-AAD using a FITC Annexin V Apoptosis Detection Kit (cat #640922, Biolegend), following the manufacturer's instructions. Stained cells were analyzed using a MACSQuant Analyzer 10 Flow Cytometer (Miltenyi Biotee). FlowJo software (Tree Star) was used to analyze the data.

Purified anti-mouse CD3 antibody (cat #100314; clone 145-2C11) used for in vitro stimulation of CD8 T cells was purchased from BD Biosciences.

Anti-mouse CD3 depleting antibody (cat #BE0002; clone 17A2) and its isotype control antibody (cat #BE0090; clone LTF-2), as well as anti-mouse PD-1 blocking antibody (cat #BE0146; clone RMPI-14) and its isotype control antibody (cat #BE0089; clone 2A3), that used for in vivo animal study were purchased from the BioXCell.

### Mouse Tumor Models

The B16-OVA murine melanoma cells (obtained from the Laboratory of Pin Wang, University of Southern California, Los Angeles, USA) (Liu et al., 2014) and the MC38 murine colon adenocarcinoma cells (obtained from the Laboratory of Antoni Ribas, UCLA) (Homet Moreno et al., 2016) were cultured in high glucose (4.5 g/L) Dulbecco's modified Eagle's medium (DMEM) supplemented with 10% FBS and Penicillin-Streptomycin (Thermo Fisher Scientific) at 37° C. and with 5% CO₂.

To establish solid tumors, mice were s.c. injected above the right flank with 1×10⁶ B16-OVA or 3×10⁵ MC38 cells. Before injection, cells in log phase of growth were harvested and suspended in phosphate-buffered saline (PBS), and 50 μl of cell suspension were injected subcutaneously above the flank. Tumor size was periodically measured with a digital Vernier caliper (Thermo Fisher Scientific).

### Bone Marrow Transfer (BMT)

Bone marrow (BM) cells were prepared from femurs and tibias by flushing with 25G needles. BM cells from CrT-KO mice were administered by retro-orbital (R.O.) injection to BoyJ female recipient mice that had received 1,200 rads of total body irradiation. Control BoyJ recipient mice received BM cells from the CrT-WT littermates. In both groups, 8×10⁶ CrT-WT or CrT-KO BM cells were injected into recipient mice. BM recipient mice were housed in a sterile environment and maintained on the combined anti-biotics sulfmethoxazole and trimethoprim oral suspension (Septra; Hi-Tech Pharmacal) for 12 weeks until analysis or use for further experiments. Blood was collected by retro-orbital bleeding and analyzed by flow cytometry to confirm the reconstitution. Tumor inoculation started 12 weeks after bone marrow transfer.

### Isolation of OT1 Transgenic T Cells and Adoptive T Cell Transfer

The OT1 transgenic T cells were purified from the spleen and lymph node cells of either OT/Tg^{CrT-WT} or OT/Tg^{CrT-KO} mice (denoted as the OT1^{CrT-WT} or OT1^{CrT-KO} cells, respectively) through magnetic-activated cell sorting (MACS) using a mouse CD8 T Cell Isolation Kit (Cat #120117044, Miltenyi Biotec) according to the manufacturer's instructions. The purified OT1^{CrT-WT} or OT1^{CrT-KO} cells were then used for in vitro culture or in vivo adoptive T cell transfer studies.

For adoptive T cell transfer, BoyJ female mice (referred to as recipient mice) were injected s.c. above the right flank with 1×10⁶ B16-OVA cells. Seven days after tumor inoculation, recipient mice received 600 rads of total body irradiation, followed by retro-orbital injection of purified OVA-specific OT1 transgenic T cells (1×10⁵ OT1 T cells per mouse).

### Tumor Infiltrating Immune (TII) Cell Isolation and Analysis

Solid tumors were collected from experimental mice at the termination of a tumor experiment. Tumors were cut into small pieces and smashed against a 70 μm cell strainer (Cat #07-201-431, Corning) to prepare single cells. Immune cells were enriched through gradient centrifugation with 50% Percoll (Cat #P4937, Sigma-Aldrich) at 800×g for 30 min at room temperature without brake, followed by treatment with Tris-buffered ammonium chloride (TAC) buffer to lyse red blood cells according to a standard protocol (Cold Spring Harbor Protocols). The resulting Tils were then utilized for further analysis.

To assess gene expression, CD45⁺ immune cells were sorted from TIIs using flow cytometry then analyzed for CrT mRNA expression using qPCR. To assess T cell activation status, TIIs were analyzed for surface activation marker (CD25 and PD-1) expression using flow cytometry. To assess T cell cytotoxicity. TIIs were analyzed for intracellular Granzyme B expression using flow cytometry. To assess T cell cytokine production, Tis were stimulated with PMA (50 ng/ml)+Ionomycine (500 ng/ml) in the presence of GolgiStop (4 μl per 6 ml culture) for 4 hours, then analyzed for intracellular cytokine (IL-2 and IFN-γ) production using flow cytometry. CD8 T cells were identified by co-staining TIIs with cell surface lineage markers (gated as CD45⁺TCRβ⁺CD4-CD8⁺ cells).

### CD8 T Cell Isolation, In Vitro Culture and Analysis

Spleen and lymph node cells were harvested from experimental mice and were subjected to magnetic-activated cell sorting (MACS) using a mouse CD8 T Cell Isolation Kit (Miltenyi Biotec) according to the manufacturer's instructions. The resulting purified CD8 T cells were then used for in vitro culture and analysis.

CD8 T cells were cultured in vitro in standard T cell culture medium comprising RPMI 1640 (Cat #10040, Corning), 10% FBS (Cat #F2442, Sigma), 1% Penicillin-Streptomycin-Glutamine (Cat #10378016, Gibco), 1% MEM Non-Essential Amino Acids Solution (Cat #11140050, Gibco), 1% HEPES (Cat #15630080, Gibco), 1% Sodium Pyruvate (100 mM) (Cat #11360070, Gibco), and 0.05 mM β -Mercaptoethanol (Cat #M3148, Sigma). Unless otherwise indicated, cells were seeded at 0.5×10⁶ cells per well in 24-well plates and stimulated with plate-bound anti-CD3 (5 μg/ml) (clone 145-2C11), for up to 5 days. At indicated time point(s), cells were collected and analyzed for CrT mRNA expression using qPCR, for cell proliferation through cell counting, for viability through Annexin V/7-AAD staining followed by flow cytometry analysis, for surface activation marker (CD25) expression through surface staining followed by flow cytometry analysis, for effector molecule (Granzyme B, IL-2, and IFN-γ) production through intracellular staining followed by flow cytometry analysis, and for cytokine (IL-2 and IFN-γ) secretion through collecting cell culture supernatants followed by ELISA analysis. CrT protein expression and TCR signaling events were analyzed using western blot analysis.

In some experiments, ATP (adenosine-5′-triphosphate disodium salt hydrate, Cat #A6419, Sigma-Aldrich) was reconstituted in sterile PBS and added to T cell culture (100 μM) for two to three days along with anti-CD3 stimulation, followed by analyzing T cell surface activation marker (CD25) expression using flow cytometry, and analyzing effector cytokine (IFN-γ) secretion using ELISA. In some experiments, T cells were stimulated with anti-CD3 for 48 hours, rested at 4° C. for 2 hours, then restimulated with anti-CD3 for 30 minutes in the presence or absence of ATP supplementation (100 μM) followed by analyzing TCR signaling events using western blot.

In some other experiments, AICAR (5-aminoimidazole-4-carboxamide 1-β-D-ribofuranoside, Cat #A9978, Sigma-Aldrich), an AMPK activator, was reconstituted in DMSO and used to pretreat T cells for 30 minutes, at the concentration of 2 mM followed by 20 minutes of anti-CD3 stimulation for western blot analysis of TCR signaling events, or at the concentration of 250 μM followed by 16 hours of anti-CD3 stimulation for flow cytometry analysis of CD25 expression and ELISA analysis of IL-2 production.

For in vitro creatine supplementation experiments, creatine monohydrate (cat #C3630, Sigma-Aldrich) was reconstituted in standard T cell culture medium and added to T cell culture. T cells were stimulated with anti-CD3 for 48 hours in the presence or absence of creatine supplementation (0.5 mM), rested at 4° C. for 2 hours, then restimulated with anti-CD3 for 10 minutes in the presence or absence of creatine supplementation (0.5 mM) followed by TCR signaling events analysis using western blot.

### MIG Mock and MIG-CrT Retroviruses

MIG Mock retroviral vector was reported previously (Li et al., 2017; Smith et al., 2015). The MIG-CrT construct was generated by inserting the mouse CrT (Slc6A8) cDNA (codon-optimized; synthesized by IDT) into the MIG retroviral vector. Retroviruses were produced using HEK293.T cells following a standard calcium precipitation method (Li et al., 2017; Smith et al., 2015). For viral transduction. CD8 T cells isolated from the spleen and lymph nodes of CrT-KO mice were stimulated in vitro with plate-bound anti-CD3 (5 μg/ml) for 4 days. On days 2 and 3 following stimulation, cells were spin infected with retroviral-containing supernatants supplemented with 10 μg/ml polybrene (Cat #TR-1003-G, Millipore) for 90 min at 770 g at 30° C. On day 4, cells were collected for analysis.

mRNA Quantitative RT-PCR (qPCR) Analysis

Total RNA was isolated using TRIzol Reagent (Cat #15596018, Invitrogen, Thermo Fisher Scientific) according to the manufacturer's instructions. cDNA was prepared using a SuperScript III First-Strand Synthesis Supermix Kit (Cat #18080400, Invitrogen, Thermo Fisher Scientific). Gene expression was measured using a KAPA SYBR FAST qPCR Kit (Cat #KM4117, Kapa Biosystems) and a 7500 Real-time PCR System (Applied Biosystems) according to the manufacturers' instructions. Ube2d2 (for T cells) or Actb (for tumor cells) was used as an internal control.

### ELISA

ELISA was performed for the detection of cytokines according to a BD Biosciences protocol. The coating and biotinylated antibodies for the detection of mouse IFN-γ (coating antibody, cat #554424; biotinylated detection antibody, cat #554426) and IL-2 (coating antibody, cat #551216; biotinylated detection antibody, cat #554410) were purchased from BD Biosciences. The streptavidin-HRP conjugate (cat #18410051) was purchased from Invitrogen. Mouse IFN-γ and IL-2 standards were purchased from eBioscience. The 3,3′,5,5′-Tetramethylbenzidine (TMB; cat #51200048) substrate was purchased from KPL. The absorbance was measured at 450 nm using an Infinite M1000 microplate reader (Tecan).

### Western Blot (WB)

Total protein was extracted using RIPA lysis buffer (Thermo Fisher Scientific) supplemented with a phosphatase inhibitor cocktail (Sigma-Aldrich) and a protease inhibitor cocktail (Roche) following the manufacturers' instructions. Nuclear protein was extracted using a Nuclear Protein Extraction Kit (Thermo Fisher Scientific) following the manufacturer's instructions, or using homemade reagents (10 mM HEPES pH 7.9, 10 mM KCl, 0.34 M sucrose, 10% glycerol, 1 mM DTT, 0.1% Triton X-100, 1.5 mM MgCl2 and protease inhibitor cocktail) following a previously established protocol (Ma et al., 2019). Protein concentration was measured by a BCA assay (Cat #23228 and Cat #1859078, Thermo Fisher Scientific). Equal amounts of protein were resolved on a 12% SDS-PAGE gel and then transferred to a PVDF membrane by electrophoresis. The following anti-mouse antibodies were purchased from Cell Signaling Technology and used to blot for the protein of interest: p-Zap-70 (cat #2705S; clone 99F2); Zap-70 (cat #2717S; clone Y319); p-Lck (cat #2751S; clone Y505); Lck (cat #2752S); p-c-Jun (cat #9261S; clone S63); NFAT (cat #4389S); NF-κB p65 (cat #8242P; clone D14E12); AMPK (cat #5831T; clone D5A2); p-AMPK (cat #2535T; clone 40H9), secondary anti-mouse (cat #7076P2), and secondary anti-rabbit (cat #7074P2). Anti-mouse CrT (SLC6A8) (cat #PA5-37060) was purchased from Thermo Fisher Scientific. β-Actin (Santa Cruz Biotechnology Inc.; cat #sc-69879; clone AC-15) was used as an internal control for total protein extracts, while Lamin A (Santa Cruz Biotechnology; cat #sc-71481; clone 4A58) was used as an internal control for nuclear protein extracts. Signals were visualized with autoradiography using an ECL system (Cat #RPN2232, Thermo Fisher Scientific). Data analysis was performed using ImageJ software (NIH).

### ATP Quantification

A Luminescent ATP Detection Assay Kit (Cat #ab113849, Abcam) was utilized to quantify intracellular ATP, following the manufacturer's instructions. Total amount of ATP detected was then normalized to cell numbers.

### Creatine Quantification

A Creatine Assay Kit (Cat #ab65339, Abcam) was utilized to quantify creatine, both in vivo and in vitro, following the manufacturer's instructions. For the in vivo study, whole blood was collected (retro-orbital bleeding) from the experimental mice in a capillary tube, and the isolated serum was immediately used for the assay following the manufacturer's directions. For the in vitro study, cells were spun to remove culture media and suspended in cold PBS. Creatine was then quantified following the manufacturer's directions. The total amount of creatine detected was then normalized to cell numbers.

### In Vivo Study of Creatine Supplementation for Cancer Immunotherapy

For creatine supplementation via i.p. injection, ceatine monohydrate (cat #C3630, Sigma-Aldrich) was dissolved in sterile PBS and i.p. injected to experimental animals daily at a dose of 10.5 mg per animal per injection.

For creatine supplementation via diet, experimental animals were fed a creatine-enriched isocaloric diet which is a customized formulation based on PicoLab Rodent Diet 20 enriched in creatine (3 g/Kg diet, cat #TD.170082, Envigo Teklad Diet). The diet was designed to reflect the safe daily dose of creatine recommended for enhanced athletic performance in humans (Mayo Clinic data). Non-treated mice (Ctrl) were fed a control diet prepared in a manner similar to that of the creatine-enriched diet.

To study the effects of creatine supplementation on suppressing tumor growth, B6 mice were inoculated with B16-OVA tumor cells and monitored for tumor growth, with or without receiving creatine supplementation via i.p. injection or diet. To study the requirement of an immune system for creatine supplementation-induced anti-tumor effects, B16-OVA tumor growth was compared between B6 mice and immune-compromised NSG mice receiving i.p. supplement of creatine. To study the T cell-dependence of creatine supplementation induced anti-tumor effects, B6-OVA tumor growth was monitored and compared in B6 mice receiving i.p. injection of an anti-CD3 T cell depleting antibody (clone RMP1-14; 100 μg/mouse/injection, twice per week) or an isotype control antibody (clone LTF-2, 100 μg/mouse/injection, twice per week), with or without i.p. supplement of creatine.

To study the combination effects of creatine supplementation and PD-1/PD-L1 blockade treatment, B6 mice were inoculated with MC38 tumor cells and monitored for tumor growth; experimental mice also received i.p. supplement of creatine, as well as i.p. injection of an anti-PD-1 blocking antibody (clone RMP1-14; 300 μg/mouse/injection, twice per week) or an isotype control antibody (clone 2A3; 300 μg/mouse/injection, twice per week), alone or in combination. Tumor-free mice were maintained for three months, then challenged with MC38 tumor cells again and monitored for tumor recurrence over another 6-month period.

### Histological Analysis

Skeletal muscle (biceps femoris) harvested from control (Ctrl) and experimental (Creatine ip and Creatine food) mice were fixed in 10% neutral-buffered formalin and embedded in paraffin for sectioning (5-μm thickness), followed by H&E staining using standard procedures (UCLA Translational Pathology Core Laboratory). The sections were imaged using an Olympus BX51 upright microscope equipped with a Macrofire® CCD camera (Optronics®). The muscle-fiber diameter was assessed with the use of an ImageJ software (NIH).

### Quantification and Statistical Analysis

FlowJo software (Tree Star) was used for the analysis of FACS data. ImageJ (NIH) was used to quantify western blots and muscle H/E sections. GraphPad Prism 6 (GraphPad Software) was used for graphic representation and statistical analysis of the data. Pairwise comparisons were made using a 2-tailed Student's t test. Multiple comparisons were performed using an ordinary 1-way ANOVA, followed by Tukey's multiple comparisons test. Data are presented as the mean±SEM, unless otherwise indicated. A P value of less than 0.05 was considered significant. ns, not significant; *P<0.05; **P<0.01; ***P<0.001; ****P<0.0001.

FIG. 8 shows the characterization of CrT-KO mice, the study of tumor growth in CrT-WT and CrT-KO mice without creatine supplementation, and the CrT mRNA expression in tumor-infiltrating CD8 T cell subsets. FIG. 9 shows the bone marrow transfer experiment studying whether CrT-deficiency in immune system directly impacts tumor growth. The figure also shows additional data studying the in vivo anti-tumor capacity of CrT-WT and CrT-KO OT1 transgenic T cells. FIG. 10 shows the creatine level in standard T cell culture medium, and the in vitro activation of CrT-WT and CrT-KO antigen-specific CD8 T cells. The figure also shows additional data studying CrT-KO CD8 T cells transduced with MIG-CrT retrovector. FIG. 11 shows the study of CrT-WT CD8 T cell activation with ATP supplementation, the study of CrT-WT and CrT-KO CD8 T cell activation with or without AICAR treatment, and the study of CrT-WT and CrT-KO CD8 T cell proximal signaling activation with or without creatine treatment. FIG. 12 shows the in vivo depletion of T cells in B6 mice using an anti-CD3 depleting antibody, and the study of CrT and Ckb mRNA expression in B16-OVA and MC38 tumor cells.

### REFERENCES

Baumeister, S. H., G. J. Freeman, G. Dranoff, and A. H. Sharpe. 2016. Coinhibitory Pathways in Immunotherapy for Cancer. Annu. Rev. Immunol. 34:539-573, https://doi.org/10.1146/annurev-immunol-032414-112049
Bengsch, B., A. L. Johnson, M. Kurachi, P. M. Odorizzi, K. E. Pauken, J. Attanasio, E. Stelekati, L. M. McLane, M. A. Paley, G. M. Delgoffe, and E. J. Wherry. 2016. Bioenergetic Insufficiencies Due to Metabolic Alterations Regulated by the Inhibitory Receptor PD-1 Are an Early Driver of CD8(+) T Cell Exhaustion. Immunity 45:358-373. https://doi.org/10.1016/j.immuni.2016.07.008
Chang, C. H., and E. L. Pearce. 2016. Emerging concepts of T cell metabolism as a target of immunotherapy. Nat. Immunol. 17:364-368. https://doi.org/10.1038/ni.3415
Chang, C. H., J. Qiu, D. O'Sullivan, M. D. Buck, T. Noguchi, J. D. Curtis, Q. Chen, M. Gindin, M. M. Gubin, G. J. van der Windt, E. Tonc, R. D. Schreiber, E. J. Pearce. and E. L. Pearce. 2015. Metabolic Competition in the Tumor Microenvironment Is a Driver of Cancer Progression. Cell 162:1229-1241. https://doi.org/10.1016/j.cell.2015.08.016
Couzin-Frankel, J. 2013. Breakthrough of the year 2013. Cancer immunotherapy. Science 342:1432-1433. https://doi.org/10.1126/science.342.6165.1432
de Campos-Ferraz, P. L., I. Andrade, W. das Neves, I. Hangai, C. R. Alves, and A. H. Lancha, Jr. 2014. An overview of amines as nutritional supplements to counteract cancer cachexia. J Cachexia Sarcopenia Muscle 5:105-110. https://doi.org/10.1007/s13539-014-0138-x
Fox, C. J., P. S. Hammerman, and C. B. Thompson. 2005. Fuel feeds function: energy metabolism and the T-cell response. Nat. Rev. Immunol. 5:844-852. https://doi.org/10.1038/nril710
Gubin, M. M., X. Zhang, H. Schuster, E. Caron, J. P. Ward, T. Noguchi, Y. Ivanova, J. Hundal, C. D. Arthur. W. J. Krebber, G. E. Mulder, M. Toebes, M. D. Vesely, S. S. Lam, A. J. Korman, J. P. Allison, G. J. Freeman, A. H. Sharpe, E. L. Pearce, T. N. Schumacher. R. Aebersold, H. G. Rammensee, C. J. Melief, E. R. Mardis, W. E. Gillanders, M. N. Artyomov, and R. D. Schreiber. 2014. Checkpoint blockade cancer immunotherapy targets tumor-specific mutant antigens. Nature 515:577-581. https://doi.org/10.1038/nature13988
Hardie, D. G., F. A. Ross, and S. A. Hawley. 2012. AMPK: a nutrient and energy sensor that maintains energy homeostasis. Nat. Rev. Mol. Cell Biol. 13:251-262. https://doi.org/10.1038/nrm3311
Ho, P. C., and S. M. Kaech. 2017. Reenergizing T cell anti-tumor immunity by harnessing immunometabolic checkpoints and machineries. Curr. Opin. Immunol. 46:38-44. https://doi.org/i0.1016/j.coi.2017.04.003
Homet Moreno, B., J. M. Zaretsky, A. Garcia-Diaz, J. Tsoi, G. Parisi, L. Robert, K. Meeth, A. Ndoye, M. Bosenberg, A. T. Weeraratna, T. G. Graeber, B. Comin-Anduix, S. Hu-Lieskovan, and A. Ribas. 2016. Response to Programmed Cell Death-1 Blockade in a Murine Melanoma Syngeneic Model Requires Costimulation, CD4, and CD8 T Cells. Cancer Immunol. Res. 4:845-857. https://doi.org/10.1158/2326-6066.CIR-16-0060
Kidani, Y., and S. J. Bensinger. 2017. Reviewing the impact of lipid synthetic flux on Th17 function. Curr. Opin. Immunol. 46:121-126. https://doi.org/10.1016/j.coi.2017.03.012
Kishton, R. J., M. Sukumar, and N. P. Restifo. 2017. Metabolic Regulation of T Cell Longevity and Function in Tumor Immunotherapy. Cell Metab. 26:94-109. https://doi.org/10.1016/j.cmet.2017.06.016
Kreider, R. B., D. S. Kalman, J. Antonio, T. N. Ziegenfuss, R. Wildman, R. Collins, D. G. Candow, S. M. Kleiner, A. L. Almada, and H. L. Lopez. 2017. International Society of Sports Nutrition position stand: safety and efficacy of creatine supplementation in exercise, sport, and medicine. J. Int. Soc. Sports Nutr. 14:18. https://doi.org/10.1186/s12970-017-0173-z
Kristensen, C. A., N. Askenasy, R. K. Jain, and A. P. Koretsky. 1999. Creatine and cyclocreatine treatment of human colon adenocarcinoma xenografts: 31P and 1H magnetic resonance spectroscopic studies. Br. J. Cancer 79:278-285. https://doi.org/10.1038/sj.bjc.6690045
Li, B., X. Wang, I. Y. Choi, Y. C. Wang, S. Liu, A. T. Pham, H. Moon, D. J. Smith, D. S. Rao, M. P. Boldin, and L. Yang. 2017. miR-146a modulates autoreactive Th17 cell differentiation and regulates organ-specific autoimmunity. J. Clin. Invest. 127:3702-3716. https://doi.org/10.1172/JCI94012
Lim, W. A., and C. H. June. 2017. The Principles of Engineering Immune Cells to Treat Cancer. Cell 168:724-740. https://doi.org/10.1016/j.cell.2017.01.016
Liu, Y., L. Xiao, K. I. Joo, B. Hu, J. Fang, and P. Wang. 2014. In situ modulation of dendritic cells by injectable thermosensitive hydrogels for cancer vaccines in mice. Biomacromolecules 15:3836-3845. https://doi.org/10.1021/bm501166j
Ma, E. H., M. C. Poffenberger, A. H. Wong, and R. G. Jones. 2017. The role of AMPK in T cell metabolism and function. Curr. Opin. Immunol. 46:45-52. https://doi.org/10.1016/j.coi.2017.04.004
Ma, X., N. K. Das, C. Castillo. A. Gourani, A. O. Perekatt, M. P. Verzi, and Y. M. Shah. 2019. SMAD family member 3 (SMAD3) and SMAD4 repress HIF2alpha-dependent iron-regulatory genes. J. Biol. Chem. 294:3974-3986. https://doi.org/10.1074/jbc.RA118.005549
McCarthy, S. A., R. A. Mufson, E. J. Pearce, J. C. Rathmell, and T. K. Howcroft. 2013. Metabolic reprogramming of the immune response in the tumor microenvironment. Cancer Biol. Ther. 14:315-318. https://doi.org/10.4161/cbt.23616
Miller, E. E., A. E. Evans, and M. Cohn. 1993. Inhibition of rate of tumor growth by crcatine and cvclocreatine. Proc. Natl. Acad. Sci. U.S.A. 90:3304-3308. https://doi.org/10.1073/pnas.90.8.3304
Nguyen, L. T., and P. S. Ohashi. 2015. Clinical blockade of PD1 and LAG3-potential mechanisms of action. Nat. Rev. Immunol. 15:45-56. https://doi.org/10.1038/nri3790
O'Neill, L. A., R. J. Kishton, and J. Rathmell. 2016. A guide to immunometabolism for immunologists. Nat. Rev. Immunol. 16:553-565. https://doi.org/10.1038/nri.2016.70
Page, D. B., M. A. Postow, M. K. Callahan, J. P. Allison, and J. D. Wolchok. 2014. Immune modulation in cancer with antibodies. Annu. Rev. Med. 65:185-202. https://doi.org/10.1146/annurev-med-092012-112807
Pardoll, D. M. 2012. The blockade of immune checkpoints in cancer immunotherapy. Nat. Rev. Cancer 12:252-264. https://doi.org/10.1038/nrc3239
Patel, C. H., and J. D. Powell. 2017. Targeting T cell metabolism to regulate T cell activation, differentiation and function in disease. Curr. Opin. Immunol. 46:82-88. https://doi.org/10.1016/j.coi.2017.04.006
Rao, E., Y. Zhang, Q. Li, J. Hao, N. K. Egilmez, J. Suttles, and B. Li. 2016. AMPK-dependent and independent effects of AICAR and compound C on T-cell responses. Oncotarget 7:33783-33795. https://doi.org/10.18632/oncotarget.9277
Ribas, A. 2015. Releasing the Brakes on Cancer Immunotherapy. N. Engl. J. Med. 373:1490-1492. https://doi.org/10.1056/NEJMp1510079
Riesberg, L. A., S. A. Weed, T. L. McDonald, J. M. Eckerson, and K. M. Drescher. 2016. Beyond muscles: The untapped potential of creatine. Int. Immunopharmacol. 37:31-42. https://doi.org/10.1016/j.intimp.2015.12.034
Rosenberg, S. A., and N. P. Restifo. 2015. Adoptive cell transfer as personalized immunotherapy for human cancer. Science 348:62-68. https://doi.org/10.1126/scicnce.aaa4967
Scharping, N. E., A. V. Menk, R. S. Moreci, R. D. Whetstone, R. E. Dadey, S. C. Watkins, R. L. Ferris, and G. M. Delgoffe. 2016. The Tumor Microenvironment Represses T Cell Mitochondrial Biogenesis to Drive Intratumoral T Cell Metabolic Insufficiency and Dysfunction. Immunity 45:374-388. https://doi.org/10.1016/j.immuni.2016.07.009
Siska, P. J., and J. C. Rathmell. 2015. T cell metabolic fitness in antitumor immunity. Trends Immunol. 36:257-264. https://doi.org/10.1016/j.it.2015.02.007
Skelton, M. R., T. L. Schaefer, D. L. Graham, T. J. Degrauw, J. F. Clark, M. T. Williams, and C. V. Vorhees. 2011. Creatine transporter (CrT; Slc6a8) knockout mice as a model of human CrT deficiency. PLoS One 6:e16187. https://doi.org/10.1371/joumal.pone.0016187
Smith, D. J., S. Liu. S. Ji, B. Li, J. McLaughlin, D. Cheng, O. N. Witte, and L. Yang. 2015. Genetic engineering of hematopoictic stem cells to generate invariant natural killer T cells. Proc. Natl. Acad. Sci. U.S.A. 112:1523-1528. https://doi.org/10.1073/pnas.1424877112
Tamas, P., S. A. Hawley. R. G. Clarke, K. J. Mustard, K. Green, D. G. Hardie, and D. A. Cantrell. 2006. Regulation of the energy sensor AMP-activated protein kinase by antigen receptor and Ca2+ in T lymphocytes. J. Exp. Med. 203:1665-1670. https://doi.org/10.1084/jem.20052469
Wherry, E. J., and M. Kurachi. 2015. Molecular and cellular insights into T cell exhaustion. Nat. Rev. Immunol. 15:486499. https://doi.org/10.1038/nri3862
Wyss, M., and R. Kaddurah-Daouk. 2000. Creatine and creatinine metabolism. Physiol. Rev. 80:1107-1213. https://doi.org/10.1152/physrev.2000.80.3.1107
Zeng. H., and H. Chi. 2017. mTOR signaling in the differentiation and function of regulatory and effector T cells. Curr. Opin. Immunol. 46:103-111. https://doi.org/10.1016/j.coi.2017.04.005

### CONCLUSION

This concludes the description of the illustrative embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

All publications mentioned herein are incorporated herein by reference to disclose and describe aspects, methods and/or materials in connection with the cited publications.

1. A composition of matter comprising:
a chemotherapeutic agent;
creatine; and
a pharmaceutically acceptable carrier.

2. The composition of claim 1, wherein creatine is present in the composition in amounts of at least 100 mg.

3. The composition of claim 1, wherein creatine is present in the composition in amounts such that:
concentrations of creatine available for tumor-infiltrating CD8 T cells are increased by at least 25% in an individual administered the composition; and/or
creatine concentrations are increased by at least 25 μM, at least 50 μM, at least 75 μM or at least 100 μM in an individual administered the composition.

4. The composition of claim 1, wherein the chemotherapeutic agent comprises an antibody.

5. The composition of claim 1, wherein the chemotherapeutic agent comprises:
carboplatin;
paclitaxel; or
at least one immune checkpoint inhibitor selected to affect a PD-1/PD-L1 blockade.

6. The composition of claim 5, wherein the checkpoint inhibitor comprises an anti-PD-1 blocking antibody and/or an anti-PD-L1 blocking antibody.

7. The composition of claim 4, wherein the antibody comprises at least one of:
pembrolizumab;
nivolumab;
atezolizumab;
avelumab;
bevacizumab; and
durvalumab.

8. A method of modulating energy metabolism in a population of tumor-infiltrating CD8 T cells comprising introducing amounts of creatine in the environment in which the CD8 T cells are disposed so that increased amounts of creatine are available for tumor-infiltrating CD8 T cell energy metabolism, thereby modulating energy metabolism in the population of tumor-infiltrating CD8 T cells.

9. The method of claim 8, wherein the tumor-infiltrating CD8 T cells are disposed in an individual diagnosed with cancer.

10. The method of claim 9, wherein the individual is undergoing a therapeutic regimen comprising the administration of a chemotherapeutic agent.

11. The method of claim 9, wherein amounts of creatine administered to the individual are selected so that concentrations of creatine available for tumor-infiltrating CD8 T cells are increased by at least 25%.

12. The method of claim 9, wherein amounts of creatine administered to the individual are selected to be at least 100 mg to at least 20,000 mg.

13. The method of claim 9, wherein amounts of creatine are selected to reduce proportions of “exhaustion-prone” phenotype cells (PD-1^{hi}CD62L^{lo}) present in a population of tumor-infiltrating CD8 T cells within the individual.

14. The method of claim 9, wherein the cancer is a lymphoma or a skin, breast, ovarian, prostate, colorectal or lung cancer.

15. The method of claim 9, wherein the tumor-infiltrating CD8 T cells are observed to exhibit:
upregulated expression of a creatine transporter gene (SLC6A8 or Crt); and/or.
impeded activation of the TCR proximal signalling molecule Zap70.

16. A method of reducing amounts of PD-1^{hi}CD62L^{lo} tumor-infiltrating CD8 T cells among a population of tumor-infiltrating CD8 T cells, the method comprising:
delivering amounts of creatine to the tumor-infiltrating CD8 T cells so that additional creatine is available for tumor-infiltrating CD8 T cell energy metabolism and amounts of PD-1^{hi}CD62L^{lo} tumor-infiltrating CD8 T cells within the population of tumor-infiltrating CD8 T cells are thereby reduced.

17. The method of claim 16, wherein the PD-1^{hi}CD62L^{lo} tumor-infiltrating CD8 T cells are within an individual diagnosed with cancer.

18. The method of claim 17, wherein the individual is undergoing a therapeutic regimen comprising the administration of at least one immune checkpoint inhibitor selected to affect a PD-1/PD-L1 blockade.

19. The method of claim 17, wherein amounts of creatine delivered to the tumor-infiltrating CD8 T cells are selected so that concentrations of creatine available for tumor-infiltrating CD8 T cells are increased by at least 25%.

20. The method of claim 17, wherein amounts of creatine administered to the individual are selected to be at least 100 mg to at least 20,000 mg.
